(19) United States
(12) Patent Application Publication
(10) Pub. No.: US 2026/0276620 A1
(43) Pub. Date: Sep. 17, 2026

(54) SYSTEMS AND METHODS FOR EVALUATING MULTIPHASE MIXTURES

(71) Applicant: Schlumberger Technology Corporation, Sugar Land, TX (US)

(72) Inventors: Linyuan Zhan, Singapore (SG); Massimiliano Fiore, Singapore (SG); Tianhang Zhang, Singapore (SG)

(21) Appl. No.: 19/075,914

(22) Filed: Mar. 11, 2025

(51) Int. Cl. G01N 33/28; G01N 27/12; G01N 27/22

(52) U.S. Cl. CPC G01N 33/2823; G01N 27/122; G01N 27/228

(57) ABSTRACT

A method of determining salinity includes isolating a reference water phase from a multiphase mixture to identify a reference salinity value, and generating a reference oil-water line for the reference salinity value with a multiphase oil-water salinity model that relates conductivity and permittivity for multiphase oil-water mixtures. With a microwave sensor, the multiphase mixture is sampled to determine a plurality of sample mixture conductivity values and sample mixture permittivity values. A sample sand-water line for the multiphase mixture is generated from the plurality of sample mixture conductivity and permittivity values. The multiphase sand-water salinity model is generated from the multiphase oil-water salinity model and a sand permittivity offset of the sample sand-water line. With the microwave sensor, an active mixture permittivity value and an active mixture conductivity value of the multiphase mixture is measured to determine an active salinity value with the multiphase sand-water salinity model.

### BACKGROUND OF THE DISCLOSURE

Wellbores may be drilled into a surface location or seabed for a variety of exploratory or extraction purposes. For example, a wellbore may be drilled to access fluids, such as liquid and gaseous hydrocarbons, stored in subterranean formations and to extract the fluids from the formations. Wellbores used to produce or extract fluids may be formed in earthen formations using earth-boring tools such as drill bits for drilling wellbores and reamers for enlarging the diameters of wellbores.

In some cases, fluids may be circulated, produced, and/or otherwise made to flow within a wellbore. Flowmeters may be implemented to measure a flowrate of these fluids. In some cases, multiphase flowmeters may measure the fractional flowrates of the various phases within a flow of a multiphase mixture. The ability for multiphase flowmeters to identify the fractional composition of a multiphase mixture may be dependent on the salinity of a brine, or a water phase, of the mixture. Accordingly, multiphase flowmeters which can account for salinity may be advantageous.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1-1 is an example of a wellbore system, according to at least one embodiment of the present disclosure;

FIG. 1-2 is another representation of the wellbore system 100, according to at least one embodiment of the present disclosure;

FIG. 2-1 illustrates an example environment in which a multiphase flowrate system is implemented in accordance with one or more embodiments described herein;

FIG. 2-2 illustrates an example implementation of a multiphase flowrate system as described herein, according to at least one embodiment of the present disclosure;

FIG. 3 illustrates an example of plot of a (σ-ε) parameter space as defined by the relationship of conductivity and permittivity in various multiphase oil-water mixtures, according to at least one embodiment of the present disclosure;

FIG. 4-1 illustrates a plot of a (σ-ε) parameter space, according to at least one embodiment of the present disclosure;

FIG. 4-2 illustrates a plot of the (σ-ε) parameter space of FIG. 4-1, according to at least one embodiment of the present disclosure;

FIG. 5 illustrates an example plot which illustrates a validation of the water salinity techniques described herein, according to at least one embodiment of the present disclosure;

FIG. 6-1 illustrates an example multiphase attenuation model for a multiphase mixture, according to at least one embodiment of the present disclosure;

FIG. 6-2 illustrates an example of an updated multiphase attenuation model according to at least one embodiment of the present disclosure;

FIG. 7 illustrates an example of an updated multiphase attenuation model, according to at least one embodiment of the present disclosure;

FIG. 8 illustrates a flow diagram for a method 800 or a series of acts for determining salinity of a flow of a multiphase mixture flowing from a wellbore as described herein, according to at least one embodiment of the present disclosure;

FIG. 9 illustrates a flow diagram for a method 900 or a series of acts for determining salinity of a flow of a multiphase mixture flowing from a wellbore as described herein, according to at least on embodiment of the present disclosure; and

FIG. 10 illustrates certain components that may be included within a computing system.

### DETAILED DESCRIPTION

This disclosure generally relates to flows of multiphase mixtures, such as that present within wellbores. A computer-implemented multiphase flowrate system is described herein which may implement various techniques for determining changes to salinity of a multiphase mixture, and in some cases, determining a multiphase flowrate of the multiphase mixture, including individual or fraction phase flowrates of specific phases in the multiphase mixture. In some cases, the present disclosure describes techniques for implementing multiphase oil-water salinity models which may apply specifically to oil-water mixtures and adapting these models to apply to sand-water mixtures (e.g., containing no oil) for the purpose of determining a salinity of the water phase. For example, in some cases a multiphase sand-water salinity model is adapted or generated from the oil-water salinity model which may facilitate determining salinity of sand-water (or sand-water-oil) mixtures notwithstanding the effects that the sand phase has on conductivity and/or permittivity measurements, upon which these multiphase oil-water salinity models rely. In this way, known relationships and/or empirical trends which may apply to oil-water mixtures may be utilized for analyzing the salinity of sand-water mixtures, which may exhibit one or different properties and for which various assumptions of oil-water mixtures may not necessarily hold true.

In some cases, the present disclosure describes techniques for implementing multiphase attenuation models for determining the proportional amount of the various phases in a multiphase mixture, for the purpose of determining the phase flowrates of the individual phases. These multiphase attenuation models may relate high- and low-energy gamma ray attenuations to the phase composition of a multiphase mixture, which attenuations may be influenced by changes in salinity of the water phase. In some cases, the multiphase attenuation model may be updated to account for an identified change to the salinity in order that the multiphase flow may be accurately determined in real time based on current and/or changing salinity of the water phase. Further, in some cases the specific salt species of the salinity may be determined and accounted for in updating the multiphase attenuation models to provide further accuracy to the determined multiphase flowrates.

As will be discussed in further detail below, the present disclosure includes a number of practical applications having features described herein that provide benefits and/or solve problems associated with evaluating multiphase flowrates. Some example benefits are discussed herein in connection with various features and functionalities provided by a multiphase flowrate system implemented on one or more computing devices. It will be appreciated that benefits explicitly discussed in connection with one or more embodiments described herein are provided by way of example and are not intended to be an exhaustive list of all possible benefits of the multiphase flowrate system.

The techniques described herein may be implemented with existing and/or conventional flowrate systems or flowmeters. For example, as described below, salinity may be determined for oil-water mixtures based on known models, relationships, and trends. The techniques herein provide flexibility by adapting these known models to advantageously apply to sand-water mixtures despite such mixtures exhibiting various properties which are not consistent with the known relationships and trends. Accordingly, the present techniques provide adaptability and flexibility for identifying salinity for sand-water mixture while utilizing systems and/or flowrates which may be otherwise calibrated in a conventional way, for example, to uniquely apply to oil-water mixtures.

Similarly, techniques are described herein which may adapt existing systems and/or products (e.g., flowmeters) which may be calibrated and/or configured to adjust for salinity, but only under the assumption that salinity is resultant from NaCl salt in the water phase. The present techniques provide an advantageous solution for identifying and accounting for different salt species compositions in the water phase, while utilizing the NaCl-adjusted flowmeters that may be existing. In this way, current and/or known systems and flowmeters may be utilized, and the present techniques may be applied thereto to increase accuracy for determining phase fraction flowrates based on accounting for a variety of salt species rather than only NaCl.

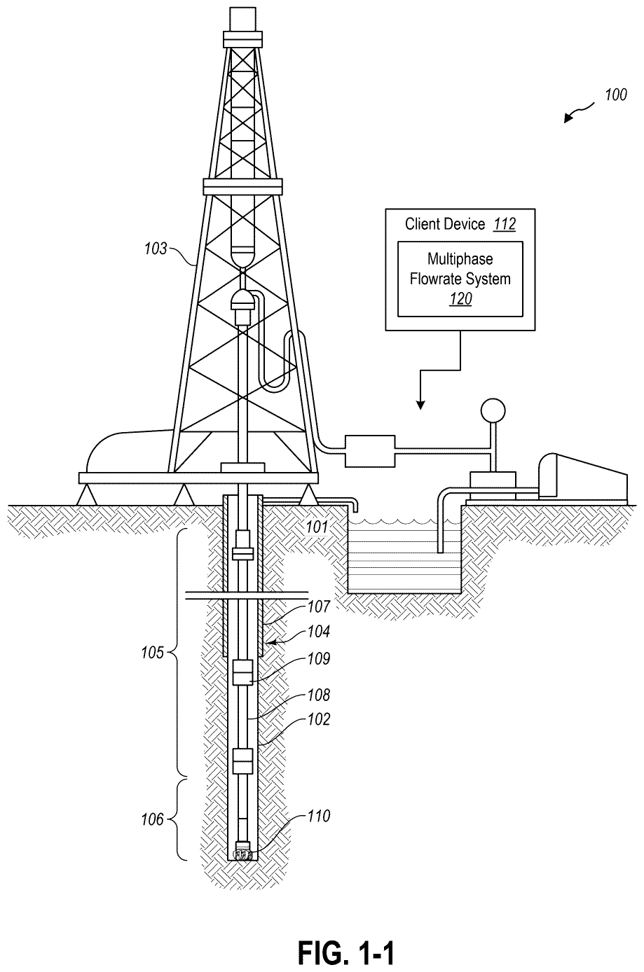

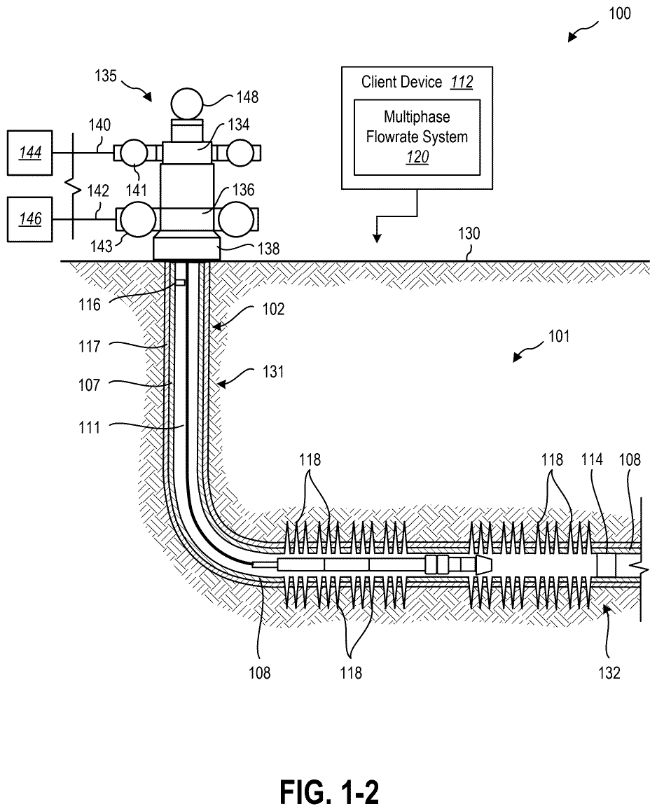

Additional details will now be provided regarding systems described herein in relation to illustrative figures portraying example implementations. For example, FIG. 1-1 shows one example of a wellbore system 100 for drilling an earth formation 101 to form a wellbore 102. The wellbore system 100 includes a drill rig 103 used to turn a drilling tool assembly 104 which extends downward into the wellbore 102. The drilling tool assembly 104 may include a drill string 105, a bottomhole assembly (“BHA”) 106, and a bit 110, attached to the downhole end of the drill string 105.

The drill string 105 may include several joints of drill pipe 108 connected end-to-end through tool joints 109. The drill string 105 transmits drilling fluid through a central bore and transmits rotational power from the drill rig 103 to the BHA 106. In some embodiments, the drill string 105 further includes additional downhole drilling tools and/or components such as subs, pup joints, etc. The drill pipe 108 provides a hydraulic passage through which drilling fluid is pumped from the surface. The drilling fluid discharges through selected-size nozzles, jets, or other orifices in the bit 110 for the purposes of cooling the bit 110 and cutting structures thereon, and for lifting cuttings out of the wellbore 102 as it is being drilled.

The BHA 106 may include the bit 110, other downhole drilling tools, or other components. An example BHA 106 may include additional or other downhole drilling tools or components (e.g., coupled between the drill string 105 and the bit 110). Examples of additional BHA components include drill collars, stabilizers, measurement-while-drilling (“MWD”) tools, logging-while-drilling (“LWD”) tools, downhole motors, underreamers, section mills, hydraulic disconnects, jars, vibration or dampening tools, other components, or combinations of the foregoing.

In general, the wellbore system 100 may include other downhole drilling tools, components, and accessories such as special valves (e.g., kelly cocks, blowout preventers, and safety valves). Additional components included in the wellbore system 100 may be considered a part of the drilling tool assembly 104, the drill string 105, or a part of the BHA 106, depending on their locations in the wellbore system 100.

The bit 110 in the BHA 106 may be any type of bit suitable for degrading downhole materials. For instance, the bit 110 may be a drill bit suitable for drilling the earth formation 101. Example types of drill bits used for drilling earth formations are fixed-cutter or drag bits. In other embodiments, the bit 110 may be a mill used for removing metal, composite, elastomer, other materials downhole, or combinations thereof. For instance, the bit 110 may be used with a whipstock to mill into casing 107 lining the wellbore 102. The bit 110 may also be a junk mill used to mill away tools, plugs, cement, other materials within the wellbore 102, or combinations thereof. Swarf or other cuttings formed by use of a mill may be lifted to the surface or may be allowed to fall downhole. The bit 110 may include one or more cutting elements for degrading the earth formation 101.

The BHA 106 may further include a rotary steerable system (RSS). The RSS may include directional drilling tools that change a direction of the bit 110, and thereby the trajectory of the wellbore. At least a portion of the RSS may maintain a geostationary position relative to an absolute reference frame, such as one or more of gravity, magnetic north, or true north. Using measurements obtained with the geostationary position, the RSS may locate the bit 110, change the course of the bit 110, and direct the directional drilling tools on a projected trajectory. The RSS may steer the bit 110 in accordance with or based on a trajectory for the bit 110. For example, a trajectory may be determined for directing the bit 110 toward one or more subterranean targets such as an oil or gas reservoir.

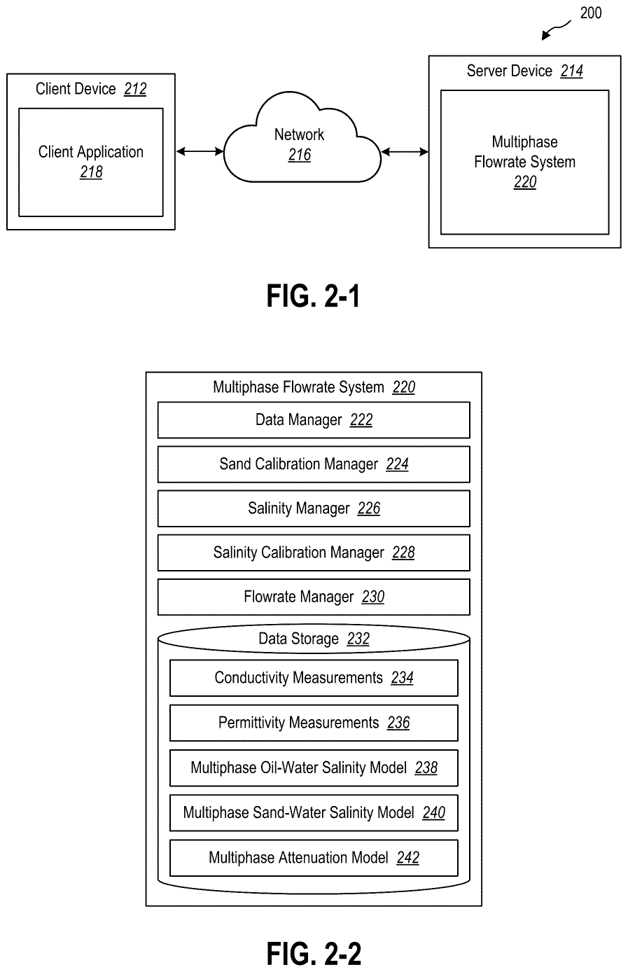

FIG. 1-2 is another representation of the wellbore system 100, according to at least one embodiment of the present disclosure. For example, FIG. 1-2 illustrates a later process or phase of the wellbore system 100 after the wellbore 102 has been (partially or completely) formed. The wellbore 102 may include a vertical portion 131 and a horizontally-extending or deviated portion 132.

The wellbore 102 may include one or more sections including one or more perforations 118. The perforations 118 may be formed through the casing 107, the cement 117 (if present), and at least a portion of the earth formation 101. The perforations 118 may be formed with a perforating gun configured to fire shaped charges into the casing 107 at desired location to penetrate the casing 107, the surrounding cement 117, and portions of the earth formation 101 surrounding the perforating gun. In some embodiments, the wellbore 102 and the earth formation 101 may be perforated in stages (sections) to form the perforations 118. In some embodiments, after perforating a section of the wellbore 102 and earth formation 101, a frac plug 114 may be positioned in the wellbore 102, configured to block the flow of fracturing fluids of the next stage so that the fracturing fluids are forced through the perforations 118. In other words, the frac plug 114 may be placed, sized, and shaped to seal lower portions of the wellbore 102 during future fracturing operations.

At a surface 130, the wellbore 102 may be capped by a fluid flow control system 135, such as a so-called “Christmas tree” or a “frac tree.” The fluid flow control system 135 may include flow control valves (e.g., master valves, wing valves, swab valves, etc.), spools, flow crosses (e.g., goat heads, frac heads, etc.), and fittings individually and/or collectively configured to direct and control (e.g., permit and prevent) flow of the treatment fluid into the wellbore 102 and to direct and control flow of formation fluids out of the wellbore 102. For example, the fluid flow control system 135 may include at least a first flow control device 134 and a second flow control device 136. The first flow control device 134 and the second flow control device 136 may individually include a valve. The first flow control device 134 and the second flow control device 136 may be configured to close selected tubulars or pipes, such as the casing 107 or production tubing extending within the wellbore 102, to selectively facilitate the flow of various fluids to or from the wellbore 102. In some embodiments, the fluid flow control system 135 includes a blow-out preventer (BOP) stack configured to selectively prevent the flow of formation fluids out of the wellbore 102. The fluid flow control system 135 may be directly or indirectly coupled to the top of a wellhead 138 (e.g., tubing head adapter) terminating the wellbore 102 at the surface 130.

In some embodiments, the first flow control device 134 is operably coupled to and in fluid communication with a first fluid conduit 140 to facilitate selective fluid connection between a first fluid and the wellbore 102. For example, the first flow control device 134 may be in fluid communication with the first fluid conduit 140 via a first valve 141. In some embodiments, the first flow control device 134 is configured to be in fluid communication with one or more of a pumpdown fluid, an acid (e.g., a spearhead treatment), a stimulation fluid, a completion fluid, a fracturing fluid, a corrosion inhibitor composition, another fluid to be provided to the wellbore 102, or combinations thereof. In some embodiments, the first fluid includes a fracturing fluid and a corrosion inhibitor composition. As described herein, the fracturing fluid may include a proppant (e.g., fracturing sand) that has been treated with a treatment fluid including a corrosion inhibitor.

The second flow control device 136 may be operably coupled to and in fluid communication with second fluid conduit 142 to facilitate selective fluid connection between a second fluid and the wellbore 102. For example, the second flow control device 136 may be in fluid communication with the second fluid conduit 142 via a second valve 143. The second fluid may include, for example, one or more of a pumpdown fluid, an acid, a stimulation fluid, a completion fluid, a fracturing fluid, a corrosion inhibitor composition, another fluid to be provided to the wellbore 102, or combinations thereof.

The first flow control device 134 and the first fluid conduit 140 may be in fluid communication with a first pump 144; and the second flow control device 136 and the second fluid conduit 142 may be in fluid communication with a second pump 146. The first pump 144 and the second pump 146 may be configured to provide a flow of a desired fluid to the wellbore 102.

In some embodiments, the flow control system 135 includes an access valve 148 to facilitate vertical access to the wellbore 102 by, for example, a bottomhole assembly or a tool string, which may be conveyed through the wellbore 102 via a wireline 111.

In some embodiments, after forming the perforations 118 in the casing 107, the cement 117, and the earth formation 101, the perforations 118 may be exposed to an acid solution to clean (e.g., remove) cement residues from the perforations 118. The acid solution may be provided to the wellbore 102 through, for example, the first fluid conduit 140 or the second fluid conduit 142, through the wellhead 138 and to the wellbore 102.

In some embodiments, after forming the perforations 118 and performing the acid treatment, a hydraulic fracturing fluid (also simply referred to herein as a “fracturing fluid”) may be provided to the wellbore 102 to open the perforations 118 and form fractures within the earth formation 101. The fracturing fluid may be provided to the wellbore 102 through the wellhead 138 via, for example, one of the first fluid conduit 140 or the second fluid conduit 142.

The fracturing fluid may be provided at a sufficient pressure to initiate fractures within the earth formation 101. The fracturing fluid may include water, one or more proppants, and one or more chemical additives. For example, the fracturing fluid may include proppants such as solid particles including sand, sintered bauxite, ceramic materials, resin-coated sand, or other particles sized, shaped, and configured to enter the fractures and maintain an open position of the fractures after the earth formation 101 and fractures are no longer exposed to the high pressures of the fracturing fluid. The solid particles may be sized and shaped to facilitate maintaining the openings in the fractures after the fracturing fluid is no longer circulated to the wellbore 102. In some embodiments, during the course of a fracturing process, the size of the proppant may gradually be increased to facilitate opening larger fractures as the fracturing process progresses.

In some embodiments, the proppants include sand. In some embodiments, the sand may have been mined from a location proximate the wellbore 102. As described herein, the sand may have been treated with a treatment fluid including a material formulated and configured to remove calcareous materials and other minerals from the sand. The treatment fluid may further include a corrosion inhibitor formulated and configured to reduce (e.g., prevent) corrosion of mining equipment, sand processing equipment, and/or sand transportation equipment.

The chemical additives that may be present in the fracturing fluid may include friction reducers, biocides (such as chlorine-based biocides (e.g., sodium hypochlorite (bleach), chlorine dioxide)), scale inhibitors, clay stabilizers, iron control agents, gelling agents, crosslinkers, breaker fluids, pH adjusters, and foaming agents. In some embodiments, the chemical additives constitute from about 0.5 weight percent to about 98.0 weight percent of the fracturing fluid. However, the disclosure is not so limited, and the chemical additives may constitute a different weight percent of the fracturing fluid.

The proppant may constitute from about 0.5 weight percent to about 10.0 weight percent of the fracturing fluid, such as from about 0.5 weight percent to about 1.0 weight percent, from about 1.0 weight percent to about 2.0 weight percent, from about 2.0 weight percent to about 4.0 weight percent, from about 4.0 weight percent to about 6.0 weight percent, from about 6.0 weight percent to about 8.0 weight percent, or from about 8.0 weight percent to about 10.0 weight percent of the fracturing fluid. However, the disclosure is not so limited, and the proppant may constitute a different amount of the fracturing fluid. In some embodiments, during the fracturing operation, a concentration of the proppant may be increased. For example, a concentration of the proppant in the fracturing fluid may increase as the fracturing operation progresses.

After the hydraulic fracturing process is complete, the pressure of the hydraulic fracturing fluid may be reduced. At least a portion of the proppants (e.g., sand) may remain in the fractures and prop the fractures open to facilitate an increase in permeability of the earth formation 101. For example, the sand may remain in the fractures, allowing the oil and gas to move towards the wellbore 102 after the pressure of the fracturing fluid is released. The increased permeability increases the production rates and overall yield from the hydrocarbon reservoir.

After the hydraulic fracturing process is complete, some or all of the wellbore 102 may be filled with the fracturing fluid. In some cases, the fracturing fluid (or other downhole fluid) may be circulated through the wellbore 102, for example to clear the wellbore and/or to remove the fracturing fluid. For example, in some cases, so-called “unconventional” oil and gas wells may operate a frac plug drill operation in which one or more of the frac plugs 114 may be degraded, drilled, or otherwise removed from the wellbore 102. Fluid may be circulated as part of this operation. Accordingly, fluid (e.g., the fracturing fluid) may be caused to flow up and out of the wellbore 102.

In some cases, the wellbore 102 may be equipped with a flowmeter 116. The flowmeter 116 may be positioned within the wellbore 102, such as at a wall of the wellbore, tubular within the wellbore, etc. and extending therefrom. In some cases, the flowmeter 116 may be positioned at or near the surface of the wellbore 102. For instance, in some cases, the flowmeter 116 is positioned at or near (e.g., after) the wellhead.

The flowmeter 116 may be a multi-phase flowmeter. The flowmeter 116 may include one or multiple sensors for measuring various properties of the flow of a multiphase mixture through the wellbore 102. For example, in some cases the flowmeter 116 includes one or more of a Venturi sensor, gamma ray sensor (e.g., dual energy level), microwave sensor, electrical conductivity sensor, temperature sensor, or a pressure sensor. The multi-phase flowmeter 116 may implement any of these sensors for determining a flowrate of the multiphase mixture, including determining a fractional composition and fractional flowrates of the constituent phases of the multiphase mixtures.

As shown in FIGS. 1-1 and 1-2, in some embodiments, the wellbore system 100 includes or is be associated with a client device 112 with a multiphase flowrate system 120 implemented thereon (e.g., or with a client application implemented thereon for accessing the multiphase flowrate system 120 as described herein). The multiphase flowrate system 120 may facilitate operating the flowmeter YYY to accurately measure the multiphase flowrate of the multiphase mixture, for example, based on the multiphase mixture including a sand phase and/or based on a salinity of the multiphase mixture resulting from different salt species.

FIG. 2-1 illustrates an example environment 200 in which a multiphase flowrate system 220 is implemented in accordance with one or more embodiments described herein. In some cases, the multiphase flowrate system 220 is the multiphase flowrate system 120 of FIGS. 1-2 and 1-2. As shown in FIG. 2-1, the environment 200 includes a server device 214. The server device 214 may include one or more computing devices (e.g., including processing units, data storage, etc.) organized in an architecture with various network interfaces for connecting to and providing data management and distribution across one or more client systems. As shown in FIG. 2-1, the server device 214 may be connected to and may communicate with (either directly or indirectly) a client device 212 through a network 216. The network 216 may include one or multiple networks and may use one or more communication platforms and/or technologies suitable for transmitting data. The network 216 may refer to any data link that enables transport of electronic data between devices of the environment 200. The network 216 may refer to a hardwired network, a wireless network, or a combination of a hardwired network and a wireless network. In one or more embodiments, the network 216 includes the internet. The network 216 may be configured to facilitate communication between the various computing devices via well-site information transfer standard markup language (WITSML) or similar protocol, or any other protocol or form of communication.

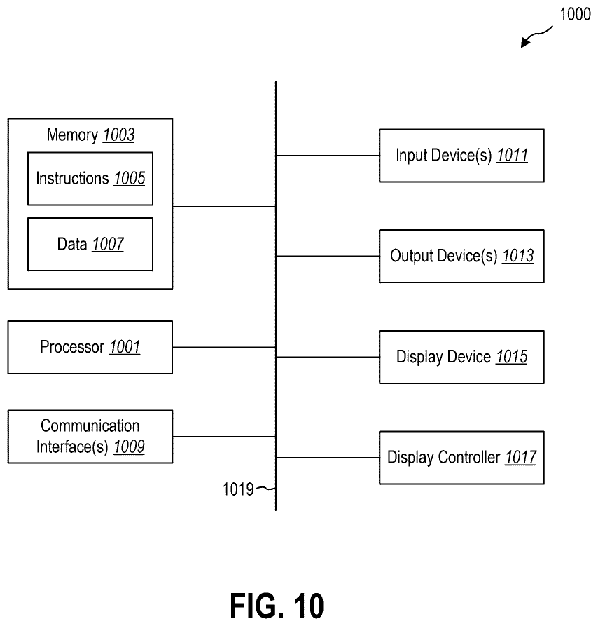

The client device 212 may be representative of one or multiple client devices and may refer to various types of computing devices. For example, the client device 212 may include a mobile device such as a mobile telephone, a smartphone, a personal digital assistant (PDA), a tablet, a laptop, or any other portable device. Additionally, or alternatively, the client device 212 may include one or more non-mobile devices such as a desktop computer, server device, surface or downhole processor or computer (e.g., associated with a sensor, system, or function of the wellbore system), or other non-portable device. In one or more implementations, the client device 212 includes graphical user interfaces (GUI) thereon (e.g., a screen of a mobile device). In addition, or as an alternative, one or more of the client device 212 may be communicatively coupled (e.g., wired or wirelessly) to a display device having a graphical user interface thereon for providing a display of system content. The server device 214 may similarly refer to various types of computing devices. Each of the devices of the environment 200 may include features and/or functionalities described below in connection with FIG. 10.

As shown in FIG. 2-1, the environment 200 may include a multiphase flowrate system 220 implemented on the server device 214. While shown on the server device 214, the multiphase flowrate system 220 may be implemented wholly or in part on the client device 212, across the server device 214 and the client device 212, or on or across one or more additional devices, such that different portions or components of the multiphase flowrate system 220 are implemented on different computing devices in the environment 200. The client device 212 may include a client application 218. The client application 218 may include an application or interface for interacting with and/or receiving the features of the multiphase flowrate system 220 as described herein. In some embodiments, one or more of the functionalities or features of the multiphase flowrate system 220 may be carried out or performed on or by the client application 218. In this way, the environment 200 may be a cloud computing environment, and the multiphase flowrate system 220 may be implemented across one or more devices of the cloud computing environment in order to leverage the processing capabilities, memory capabilities, connectivity, speed, etc., that such cloud computing environments offer in order to facilitate the features and functionalities described herein.

FIG. 2-2 illustrates an example implementation of the multiphase flowrate system 220 as described herein, according to at least one embodiment of the present disclosure. The multiphase flowrate system 220 may include a data manager 222, a sand calibration manager 224, a salinity manager 226, a salinity calibration manager 228, and a flowrate manager 230. The multiphase flowrate system 220 may also include a data storage 232 having various data stored thereon. For example, the data storage 232 may store conductivity measurements 234 and permittivity measurements 236. In some cases, a multiphase oil-water salinity model 238 and a multiphase sand-water salinity model 240 are stored via the data storage 232. In some cases, one or multiple multiphase attenuation models 242 are stored on the data storage. While one or more embodiments described herein describe features and functionalities performed by specific components 222-230 of the multiphase flowrate system 220, it will be appreciated that specific features described in connection with one component of the multiphase flowrate system 220 may, in some examples, be performed by one or more of the other components of the multiphase flowrate system 220.

By way of example, one or more data measurement, receiving, gathering, or storing features of the data manager 222 may be delegated to other components of the multiphase flowrate system 220. As another example, while the sand calibration manager 224 may facilitate updating and or generating one or more multiphase models associated with various multiphase mixtures as described herein, in some cases some or all of these features may be performed by the salinity manager 226 (or other component of the multiphase flowrate system 220). Indeed, it will be appreciated that some or all of the specific components may be combined into other components and specific functions may be performed by one or across multiple components 222-230 of the multiphase flowrate system 220.

Additionally, while FIGS. 1-1 and 1-2, for example, depict a multiphase flowrate system 120 implemented on a client device 112 of the wellbore system 120, it should be understood that some or all of the features and functionalities of the multiphase flowrate systems described herein may be implemented on or across multiple client devices and/or server devices. For example, data may be input and/or received by the data manager 222 on a (e.g., local) client device, and the one or more salinity or flowrate values may be determined on one or more of a remote, server, or cloud device. Indeed, it will be appreciated that some or all of the specific components 222-230 may be implemented on or across multiple client devices 212 and/or server devices 214, including individual functions of a specific component being performed across multiple devices.

As mentioned above, the multiphase flowrate system 220 includes a data manager 222. In some cases, the data manager 222 receives measurement data. For example, the data manager 222 may receive measurement data from one or more sensors, such as one or more sensors of the flowmeter as described above. In some cases, the measurement data includes a flowrate, such as a volumetric flowrate of a fluid (e.g., a multiphase mixture) flowing within the wellbore. The flowrate may be measured via a Venturi sensor, or other suitable sensor. The measurement data may include conductivity measurements and/or permittivity measurements of the flow of the multiphase mixtures. For example, microwave sensors such as a single microwave open coaxial reflection probe may measure the conductivity measurements and/or permittivity measurements of the multiphase mixture (and in some cases, of an isolated phase that is made to flow to the sensor).

In some cases, the measurement data includes gamma ray measurements from a gamma ray sensor. The gamma ray measurements may include high-energy gamma ray measurements and low-energy gamma ray measurements. The high-energy gamma ray measurements may be in a range of about 300 keV to about 1.5 MeV. The low-energy gamma ray measurements may be in a range of about 30 keV to about 300 keV. The high- and low-energy gamma ray measurements may facilitate characterizing the density of the multiphase mixture both as an overall density and to discriminate between particular phases of the multiphase mixture.

In some embodiments, the data manager 222 receives user input. The data manager 222 may receive the user input, for example, via any of the client devices 212 and/or server devices 214. Any of the data described herein may be input or augmented via the user input. For example, in some instances, the multiphase oil-water salinity model is received by the data manager 222 as user input. The user input may be received in association with any functions or features of the multiphase flowrate system 220.

As mentioned above, the flowmeter 116 and the multiphase flowrate system 220 may be implemented to measure and determine a flowrate of a multiphase mixture, including constituent flowrates of the various phases of the multiphase mixture. This may be accomplished based on measuring the total flowrate of the mixture and based on identifying the fractional proportions of the various phases of the mixture, and inferring phase flowrates of the individual phases. In particular, flowmeters may be utilized for measuring a water cut, or water-to-liquid ratio of the multiphase mixture.

Flowmeters may operate in this way based on utilizing dual-energy gamma ray techniques to measure and/or infer phase fractions of a multiphase mixture. For example, high-energy and low-energy gamma rays may be emitted into and received through the mixture, and the detected attenuation of the gamma rays may indicate the ratio of the specific phases. To elaborate, each phase may absorb or attenuate the gamma rays to a certain degree, which may be represented by a mass attenuation coefficient for the phase. Based on the measured attenuated gamma rays, and based on known or calibrated mass attenuation coefficients of the phases, the proportions of each phase may be determined.

In some cases, the mass attenuation coefficient of the water phase may be highly dependent on the salinity of the water phase (e.g., the water phase being a brine). For example, a changing salt content of the water phase may affect the density of the water phase, which may affect the way in which the water phase absorbed or attenuates the emitted gamma rays. Thus, as salinity changes, if the mass attenuation coefficient for the water-phase is not accordingly calibrated, the results of the multiphase flowmeter from the initial calibration values will result in erroneous water-cut and/or phase fraction flowrates. Accordingly, monitoring changes in water salinity in real time, under multiphase flow conditions is highly desirable.

In some cases, the data manager 222 receives and/or accesses a multiphase oil-water salinity model. The multiphase oil-water salinity model may facilitate characterizing the salinity of the water phase of a multiphase oil-water mixture, for example, in order to calibrate the multiphase flowmeter to changing salinity. The multiphase oil-water salinity model may be applicable to multiphase flows which substantially contain oil and water, or multiphase flows which contain oil, water, and gas. A multiphase oil-water mixture may include other constituent parts, such as solids, other liquids, etc., but in such small amounts as to be negligible with respect to the present flowrate measurement techniques, and herein may be considered substantially an oil-water (or oil-water-gas) mixture.

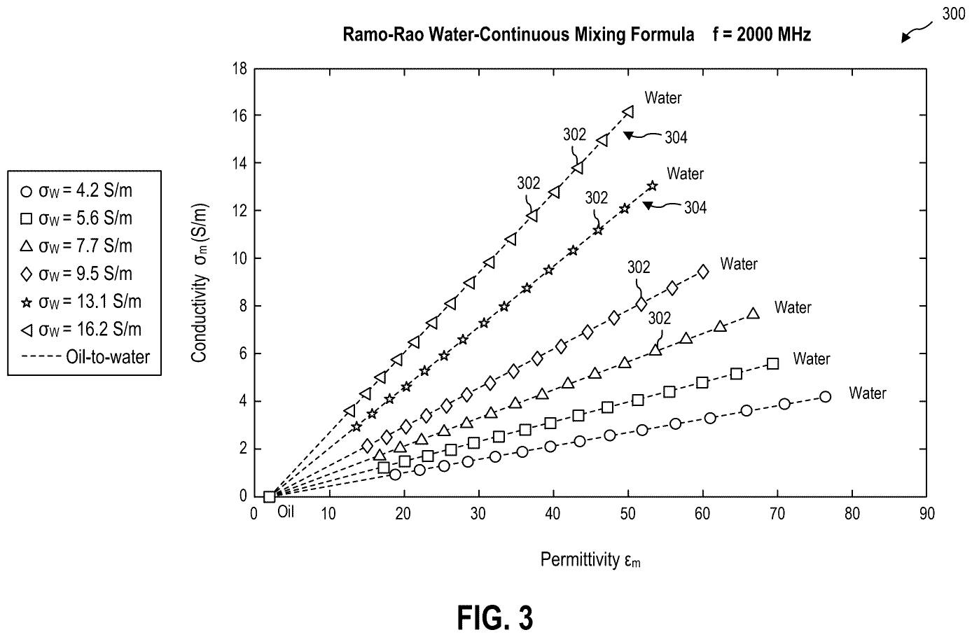

The multiphase oil-water salinity model may be a model which defines relationships of conductivity (σ) and permittivity (ε) in a (σ-ε) parameter space. In particular, the multiphase oil-water salinity model may relate conductivity and permittivity for multiphase oil-water mixtures. For example, the multiphase oil-water salinity model may be generated, derived, and/or based on empirical measurement data and based on various assumptions. FIG. 3 illustrates an example of plot 300 of a (σ-ε) parameter space as defined by the relationship of conductivity and permittivity in various multiphase oil-water mixtures, according to at least one embodiment of the present disclosure. For instance, in some cases, the plot 300 of the (σ-ε) parameter space of FIG. 3 may be representative of the multiphase oil-water salinity model.

As shown in FIG. 3, for multiphase oil-water mixtures that are water continuous (e.g., water is the continuous phase and oil is the dispersed phase), the conductivity and permittivity of a multiphase oil-water mixture tend to fall close to distinct lines connecting an oil point (e.g., 100% oil) and a water point (e.g., 100% water), when water conductivity is fixed (e.g., for a given salinity, temperature, and pressure). This is observed to be the case because, when water is rich (e.g., continuous), the mixture complex permittivity is dominated by the presence of water, whereas gas and oil (hydrocarbons) have small and similar complex permittivities. In some cases, this is also due to the fact that hydrocarbon (gas and oil) volume fraction influences mixture permittivity and mixture conductivity in the similar way when water is continuous. This characteristic trend has been observed, in some cases, to uniquely apply to water-rich (e.g., water continuous) oil-water mixtures.

The plot 300 illustrates various example (σₘ, εₘ) data points 302 corresponding to 6 oil-water lines 304 for water conductivities of σ_{w}=4.2 to 16.2 S/m. The example the (σₘ, εₘ) data points 302 range water cuts (proportion of water in mixture) from 1.0 to 0.3 with a 0.05 step (e.g., labeled at the oil-water line for mixtures with σ_{w}=4.2 S/m). Oil permittivity is approximately 2.1, and oil conductivity is 10⁻⁸ S/m (e.g., negligible, or 0). The plot 300 may be generated from the relationships defined by the multiphase oil-water salinity model, which may be based on formulae derived by Ramu and Rao for liquid/liquid mixture permittivity and mixture conductivity (σₘ, εₘ). (See Hammer E A: “Flow permittivity models and their application in multiphase meters,” Proc. Multiphase Metering, IBC Technical Services, 12-13 Mar. 1997, Aberdeen; Hammer E A: “Three component flow measurement in oil/gas/water mixtures using capacitance transducers,” Ph.D. Thesis, UMIST, U.K., 1993).

For each fixed water conductivity, as the ratio of water in the mixture varies, the (σₘ, εₘ) data points 302 follow the same slope, falling close on the associated oil-water line 304. Note that the slopes in the (σ-ε) parameter space are simply σₘ/(εₘ-εₒᵢₗ). For water-continuous flows, there are generally σ_{w}>>σₒᵢₗ and σₘ>>σₒᵢₗ. The relationship as defined by the multiphase oil-water salinity model may be based on Bruggeman's mixing laws, including:

$$\frac{{ε}_{2} - {ε}_{m}}{{ε}_{2} - {ε}_{1}} {( \frac{{ε}_{1}}{{ε}_{m}} )}^{\frac{1}{3}} = 1 - v$$

$$\frac{{σ}_{2} - {σ}_{m}}{{σ}_{2} - {σ}_{1}} {( \frac{{σ}_{1}}{{σ}_{m}} )}^{\frac{1}{3}} = 1 - v$$

Where: v is the volume fraction of the dispersed phase.

From Bruggeman's mixing laws, it can be readily derived, by assuming equal volume fractions v, that:

$$\frac{{σ}_{m}}{{ε}_{m}} = {( \frac{{S}_{m}}{{S}_{w}} )}^{3} \cdot \frac{{σ}_{w}}{{ε}_{w}}$$

Where:
Sₘ=Slope of mixture (line from mixture (σₘ, εₘ) data point(s) to oil point);
   S_{w}=Slop of water (Line from water point to oil point); and
The slopes of the mixture and single-phase water are equal (e.g., Sₘ/S_{w}=1)

Accordingly, for water-continuous mixtures:

$$\frac{{σ}_{m}}{{ε}_{m}} \approx \frac{{σ}_{w}}{{ε}_{w}}$$

The multiphase oil-water salinity model may then be based on the assumption that the ratio of the mixture conductivity to the mixture permittivity is approximately equal to the ratio of water conductivity to water permittivity, when the mixture is water continuous, which trend may be represented by the slope of an oil-water line 304 and/or set of (σₘ, εₘ) data points 302. This assumption has been observed to be substantially accurate for oil-water, and oil-water-gas multiphase mixtures. As illustrated, as the water conductivity (e.g., water salinity) changes, the slope of the corresponding oil-water line changes or rotates about the oil point. Thus, for given measurement values of mixture conductivity and mixture permittivity, the salinity of the water phase of the mixture may be deduced based on the water conductivity associated with a corresponding oil-water line as defined by the multiphase oil-water salinity model.

The assumption of the multiphase oil-water salinity model that the ratio of the mixture conductivity to the mixture permittivity is approximately equal to the ratio of water conductivity to water permittivity has been shown to be substantially true by empirical data. Indeed, for oil-water mixtures and even for oil-water-gas mixtures, when water is continuous, empirical (σₘ, εₘ) data points fall close to the oil-water lines 304 for each fixed water conductivity. This is because, as mentioned above, when water is rich, the complex reflection coefficient is dominated by the presence of water (later term in the complex permittivity), whereas the gas and oil (hydrocarbon) have small and similar complex permittivities. It is also because hydrocarbon (gas and oil) volume fraction influences σₘ and εₘ in a similar way when water is continuous.

This similarity in ratios has been shown to be true despite the oil point of the oil-water lines having a small permittivity offset. For example, the oil phase (e.g., at the oil point) may be observed to have a permittivity value. As shown, the oil-water lines 304 may not necessarily intersect the origin of the (σ-ε) perimeter space, but rather may be offset in the ε dimension based on the observed permittivity of the oil phase. The multiphase oil-water salinity model may operate based an oil relative permittivity of about 2.1 and a conductivity of 0 S/m (siemens per meter). This offset by the oil permittivity may be relatively small in this way, and accordingly the assumption may hold substantially true that the ratio of the water conductivity to the water permittivity is approximately equal to the ratio of the mixture conductivity to the mixture permittivity. Thus, despite not being an exact 1:1 ratio (i.e., due to the oil permittivity offset), in some cases, this assumption can be held to be substantially true and useful to a requisite degree as to provide highly reliable and accurate results when determining water salinity.

In some cases, the multiphase oil-water salinity model may be based on the following relationship:

$${σ}_{w} = f ( \frac{{σ}_{m}}{{ε}_{m - {ε}_{O}}} , P , T , ω )$$

Where:
σ_{w}=water conductivity
σₘ=mixture conductivity
εₘ=mixture permittivity
ε_{O}=oil permittivity
P=mixture pressure
T=mixture temperature
ω=microwave frequency

For example, the multiphase oil-water salinity model may relate the water conductivity as a function of the mixture permittivity and conductivity, and the offset of the oil permittivity, as well as the temperature, pressure, and microwave frequency at which the mixture permittivity and conductivity measurements were taken. In some cases, the multiphase oil-water salinity model may relate the mixture conductivity-permittivity trend more directly to the water salinity. For example:

$$Salinity = f ( \frac{{σ}_{m}}{{ε}_{m - {ε}_{O}}} , P , T , ω )$$

Accordingly, the slope of a set of (σₘ, εₘ) data points 302 (e.g., and corresponding oil-water lines 304) may be leveraged to form the basis for real-time water-conductivity estimates. Indeed, by accurately estimating water conductivity, the salinity of the multiphase oil-water mixture may be determined, which in turn may facilitate dynamically updating the corresponding mass attenuation coefficients for the purposes of producing accurate flowrate measurements. The multiphase oil-water salinity model is described in detail in U.S. Pat. No. 6,831,470, which is hereby incorporated by reference in its entirety.

As mentioned above, in some situations, such as during fracking or fracking-associated wellbore operations, fluid may flow from the wellbore which may include various solids therein, such as a fracking fluid. In a particular example, a multiphase mixture flowing in the wellbore may include a sand phase, which may be dispersed within the mixture to form a slurry. For instance, in some wellbore operations, such as frack-plug drilling operations, a multiphase mixture (e.g., fracturing fluid) may be circulated within the wellbore which may be substantially a sand-water mixture, or a sand-water-gas mixture. For example, the multiphase mixture may not include oil, or may only include trace amounts of oil to the extent that any included oil is not substantial enough to be considered a defined phased of a sand-water mixture. Because the multiphase mixture during these wellbore operations may comprise different constituent phases than an oil-water mixture as discussed above, in some cases, the multiphase oil-water salinity model may not be accurately applied to sand-water mixtures to determine salinity, and accordingly to accurately calibrate mass attenuation coefficients.

In some cases, the sand calibration manager 224 may facilitate utilizing the multiphase oil-water salinity model in order to accurately determine salinity in a sand-water mixture. For example, in some cases, the calibration manager 224 may generate a multiphase sand-water salinity model based on the multiphase oil-water salinity model. For example, in some cases it has been observed that while some assumptions and/or relationships of the multiphase oil-water salinity model may not be accurate with respect to sand-water mixtures, one or more other aspects may nevertheless remain true, which the calibration manager 224 may leverage in order to accurately determine salinity of a sand-water mixture based on similar principles to that of the multiphase oil-water salinity model.

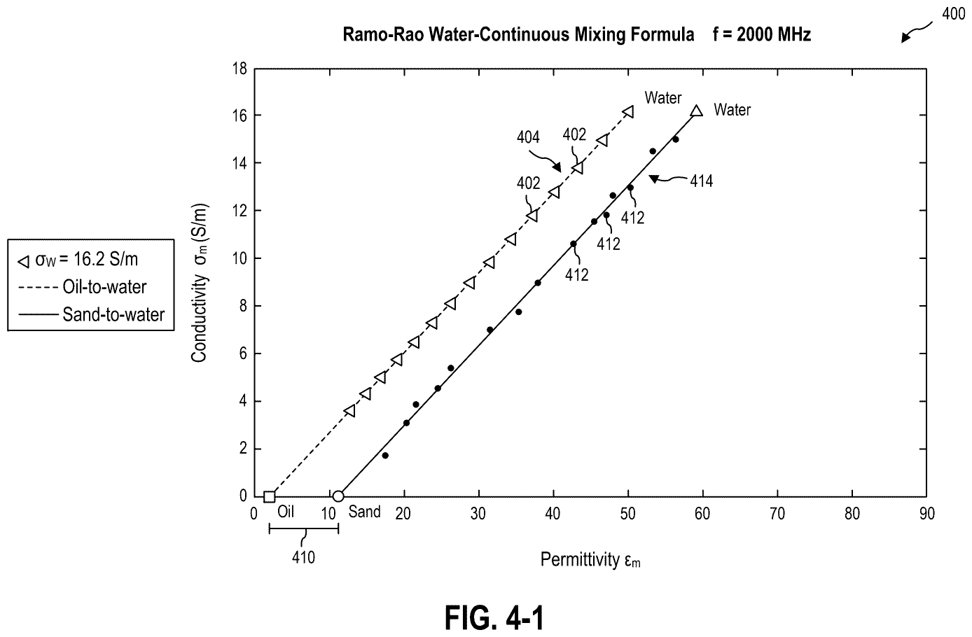

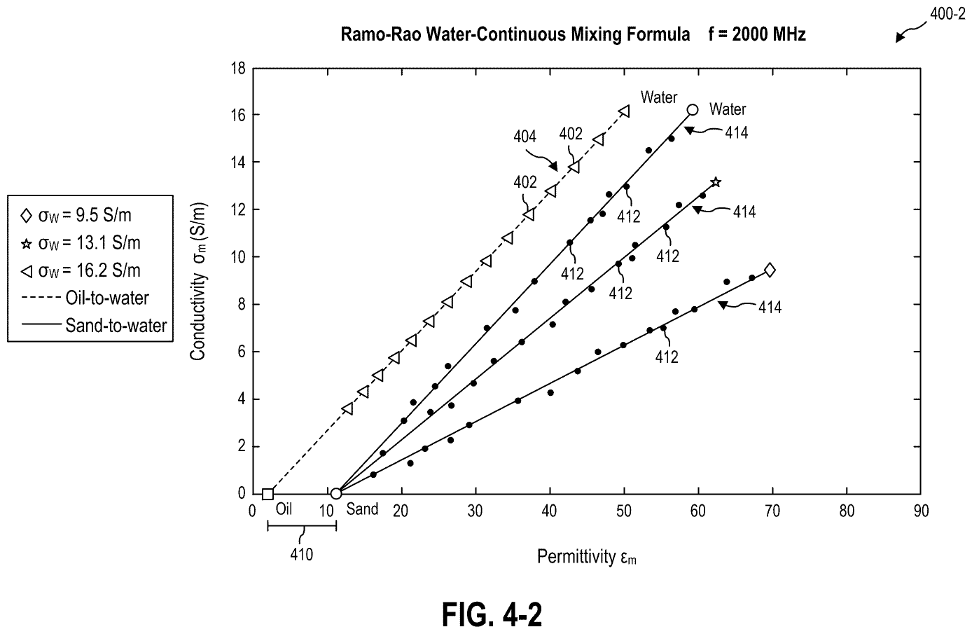

FIG. 4-1 illustrates a plot 400 of a (σ-ε) parameter space, according to at least one embodiment of the present disclosure. The (σ-ε) parameter space may be the same parameter space as that of the plot 300 of FIG. 3. For example, the plot 400 indicates an oil-water line 404 having example (σₘ, εₘ) data points 402 corresponding with a set σ_{w} (e.g., set water salinity) of 16.2 S/m. Note that, as discussed above, the oil-water line 404 and the example (σₘ, εₘ) data points 402 are applicable to an oil-water mixture. The plot 400 also includes sample (σₘ, εₘ) data points 412, representative of permittivity and conductivity measurements taken for a sand-water mixture. The sample (σₘ, εₘ) data points 412 define a sand-water line 414. For instance, the (σₘ, εₘ) data points 412 may generally lie on or are scattered about a line which, when fit to the (σₘ, εₘ) data points 412, defines the sand-water line 414. The sand-water mixture from which the sample (σₘ, εₘ) data points 412 were measured had a water phase also of σ_{w}=16.2, and accordingly the water salinity for the oil-water line 404 corresponds with the water salinity for the sand-water line 414.

As shown in the plot 400, the sample (σₘ, εₘ) data points 412 and the sand-water line 414 do not align with the oil-water line 404, illustrating the fact that the multiphase oil-water salinity model does not accurately represent sand-water mixtures. However, as can be seen in the plot 400, the slope of the sand-water line 414 is approximately equal to that of the oil-water line 404. Accordingly, the assumption of the multiphase oil-water salinity model that the ratio of water conductivity to water permittivity is approximately equal to the ratio of the mixture conductivity to the mixture permittivity (for oil-water mixtures) would also be true for sand-water mixtures, but for a sand permittivity offset 410 of the sand-water line. Indeed, similar to the oil point, the sand point is offset (e.g., from the origin) by the sand permittivity offset 410. Because the sand permittivity offset 410 of the sand point is (e.g., much larger) than that of the oil point, the sand permittivity offset 410 cannot be ignored or otherwise assumed to not affect the assumptions as discussed above. For example, as the oil point permittivity is relatively small (e.g., about 2.1) it is assumed to be negligible with respect to the mixture slope (ratio) and water slope (ratio) being equal for oil-water mixture. However, the sand relative permittivity offset 410 is considerably larger (e.g., about 10) such that it cannot be said that the ratio of the water conductivity to the water permittivity is equal to the ratio of the mixture conductivity to the mixture permittivity for sand-water mixtures. Such an assumption would, in some cases, lead to inaccurate salinity measurements. Rather, the mixture slope (e.g., ratio of mixture conductivity to mixture permittivity) is offset from the water slope (e.g., ratio of water conductivity to water permittivity) by the sand permittivity offset 410. In other words, a sand-water mixture causes the permittivity of a corresponding conductivity to be offset by the sand permittivity offset 410 from the permittivity value as defined by the oil-water line of the same salinity. Accordingly, the sand point having the sand permittivity offset 410 may be representative of one way in which the multiphase oil-water salinity model may not be entirely representative of sand-water mixtures.

FIG. 4-2 illustrates a plot 400-2 of the (σ-ε) parameter space of plot 400. The plot 400-2 includes additional sample (σₘ, εₘ) data points 412 and corresponding sand-water lines 414 for sand-water mixtures of different conductivity values (e.g., different salinity). In some cases, it is observed that the sand permittivity offset 410 remains constant for varying water conductivity values (e.g., salinity) of the sand-water mixtures. Indeed, similar to the multiphase oil-water salinity model which, for changing water conductivity values (e.g., salinity) the corresponding oil-water lines are observed to change in slope and rotate about the oil point, for changing water conductivity values (e.g., salinity) of sand-water mixtures, the corresponding sand-water lines 414 are observed to rotate about the sand point, and the sand permittivity offset 410 remains constant. In some cases, this is observed to be true as long as the sand phase of the sand-water mixture remains unchanged, such as the same type, kind, grain size, etc. of sand (e.g., while the proportion of the sand phase in the multiphase mixture may change).

In some embodiments, the sand calibration manager 224 may generate the multiphase sand-water salinity model based on identifying the sand permittivity offset 410 of a sand-water mixture. For example, for a wellbore operation in which a sand-water mixture is circulated in the wellbore, the sand calibration manager 224 may facilitate implementing a calibration period of the flow of the multiphase mixture. For instance, the sand calibration manager 224 may facilitate calibrating a flowmeter such that the flowmeter may accurately measure the multiphase flow (e.g., the fractional phase flows) based on accurately identifying salinity (and changes thereof) of the water phase of the mixture.

During a calibration period, the calibration manager 224 may receive a reference salinity value for the sand-water mixture. For example, the reference salinity value may be determined based on taking a sample of the sand-water mixture and isolating the water phase from the sample. For instance, the isolated water phase may be caused to flow to the flowmeter (e.g., to a microwave sensor) where the conductivity value (e.g., corresponding to salinity) of the single-phase water may be measured, which may be the water point conductivity. In some cases, wellbore personnel may facilitate sampling the mixture and isolating the water phase. In some cases, the isolated water phase is examined and/or tested separately from the wellbore and/or the flowmeter. For example, the isolated water phase may be examined (e.g., in situ at the wellbore site, in a lab, or elsewhere) and/or tested to determine the water conductivity and/or water salinity. For instance, the density of the isolated water phase may be measured, and the salinity determined accordingly. The reference salinity value may be a value or measure of the salinity (e.g., in parts per million (PPM)), or else may be an indication of the water conductivity value for the water phase (e.g., water point) of the measured salinity.

Based on receiving the reference salinity value, the sand calibration manager 224 may identify and/or generate a reference oil-water line. For example, the reference oil-water line may be an oil-water line (e.g., such as the oil-water line 404 in the plot 400) that corresponds with the determined water conductivity of the isolated water phase. For example, the calibration manager 224 may identify, with the multiphase oil-water salinity model, a (σ_{w}, ε_{w}) data point 402 corresponding with the reference salinity value and may generate a reference oil-water line between the (σ_{w}, ε_{w}) data point 402 and the oil point (e.g., at (0, 2.1)).

During the calibration period, the calibration manager 224 may facilitate calibration the flowmeter based on generating a sample sand-water line for the sand-water mixture. For example, the calibration manager 224 may facilitate measuring the sand-water mixture flowing in the wellbore. For example, a microwave sensor (e.g., of the flowmeter) may measure the conductivity and permittivity for a plurality of (e.g., at least two) sample periods. The calibration manager 224 may accordingly generate a plurality of (σₘ, εₘ) data points 412 in the (σ-ε) parameter space, which may correspond to ordered pairs of conductivity and permittivity for a sample of the sand-water mixture. Based on the plurality of (σₘ, εₘ) data points 412, the calibration manager 224 may construct the sample sand-water line based on fitting a line to the points.

From the sample sand-water line, the calibration manager 224 may determine the sand point, or the (σₘ, εₘ) data point corresponding with a (e.g., theoretical) mixture of a single, sand phase. For example, the calibration manager 224 may determine the permittivity value where the sand-water line intersects the permittivity axis, or the permittivity corresponding to a conductivity value of 0 for the sand-water line. Accordingly, the calibration manager 224 may determine the sand permittivity offset, representing a shift of the sample sand-water line from the origin along the permittivity axis of the (σ-ε) parameter space.

In some cases, the calibration manager 224 may determine, from the reference oil-water line, the slope of the reference oil-water line. As discussed above, the oil-water and sand-water lines for oil-water and sand-water mixtures (respectively) having the same salinity of the water phase are observed to be parallel, or to have the same slope. Accordingly, based on the determined slope of the reference oil-water line, the calibration manager 224 may, in some cases, generate the sample sand-water line using (e.g., at least one) sample (σ_{w}, ε_{w}) data point 412 from the sand-water mixture and the identified slope. The calibration manager 224 may then determine the sand permittivity offset 410 in any manner as those just discussed.

Based on determining the sand permittivity offset 410 the calibration manager 224 may generate a multiphase sand-water salinity model which may correspond with the specific sand-water mixture being circulated in the wellbore (e.g., may be uniquely applicable to a mixture having a specific kind, type, size, etc., of sand). For example, the calibration manager 224 may update one or more of the relationships, functions, formulas, equations, assumptions, or other aspects of the multiphase oil-water salinity model to account for the sand permittivity offset and generate the multiphase sand-water salinity model. In some cases, the multiphase sand-water salinity model may be based on the following relationship:

$${σ}_{w} = f ( \frac{{σ}_{m}}{{ε}_{m} - {ε}_{sand}} , P , T , ω )$$

Where:
σ_{w}=water conductivity
σₘ=mixture conductivity
εₘ=mixture permittivity
ε_{sand}=sand permittivity (sand permittivity) offset
P=mixture pressure
T=mixture temperature
ω=microwave frequency

In some cases, the multiphase sand-water salinity model may more directly relate a salinity value of the water phase, such as with the following relationship:

$${Salinity}_{with sand} = f ( \frac{{σ}_{m}}{{ε}_{m} - {ε}_{sand}} , P , T , ω )$$

Accordingly, a flowmeter of the wellbore may be calibrated to accurately measure and/or determine salinity of a sand-water mixture (and/or of a water phase of the mixture) circulating in the wellbore based on the multiphase sand-water salinity model. The multiphase sand-water salinity model may be uniquely applicable to the sand-water mixture circulating in the wellbore. Accordingly, the multiphase sand-water salinity model may be advantageously utilized for characterizing the salinity of the mixture for a duration of the wellbore operation in which the sand-water mixture is being circulated in the wellbore, for example, which may last days or weeks. In this way, the flowmeter may be calibrated (e.g., once) during an initial or calibration period (e.g., an initial period of the wellbore operation) and the multiphase sand-water salinity model may remain applicable to the sand-water mixture. For instance, the proportion of sand and water in the multiphase sand-water mixture may change, but the multiphase sand-water salinity model may be utilized for determining a (e.g., changing) salinity in the water phase, so long as the kind, type, or species of the sand phase is not significantly altered. Additionally, in some cases the same sand-water mixture may be circulated in multiple different wellbores. For example, the same kind of sand-water mixture (e.g., having the same sand species) may be prepared and circulated in several different wellbores, or in some cases, the same source of sand-water mixture may be circulated throughout multiple wellbores. In this way, the multiphase sand-water salinity model may be generated (e.g., based on one wellbore) which may facilitate calibrating flowmeters for multiple different wellbores.

In some cases, the salinity manager 226 may facilitate determining a salinity of a multiphase sand-water mixture circulating in the wellbore. For example, the salinity manager 226 may implement the multiphase sand-water salinity model as generated by the sand calibration manager 224. To elaborate, a flowmeter and/or a microwave sensor may take (e.g., continually and/or periodically) current, real time, and/or active permittivity measurements and conductivity measurements of the sand-water mixture. Based on active mixture permittivity values and active conductivity values, the salinity manager 226 may determine an active water salinity based on the multiphase sand-water salinity model. In some cases, the salinity manager 226 receives temperature and pressure measurements of the sand-water mixture for use with the sand-water multiphase flow model as described herein. The salinity manager 226 may identify and incorporate a microwave frequency of the measurement data taken by the microwave sensor.

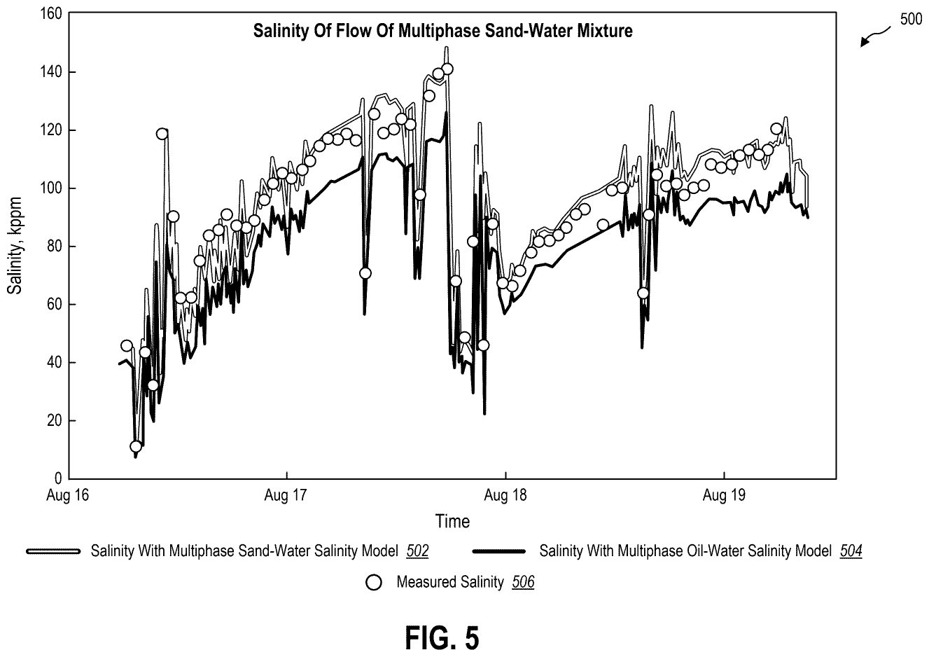

FIG. 5 illustrates an example plot 500 which illustrates a validation of the water salinity techniques described herein, according to at least one embodiment of the present disclosure. The plot 500 illustrates salinity values determined for a flow of a multiphase sand-water mixture circulating in a wellbore over the course of several days. The salinity values were determined based on the multiphase oil-water salinity model (504), as well as based on the improved techniques described herein which implement a multiphase sand-water salinity model (502). As shown in the plot 500, the salinity as determined with the multiphase sand-water salinity model 502 tracks closely with measured salinity values 506 as taken periodically over the several day validation period. The salinity as was determined in the plot 500 was based on calibrating a flowmeter as described herein during an initial, calibration period, and utilizing that calibration over the entire range of the plot 500. These improvements show the accuracy provided by the multiphase flowrate system described herein for determining salinity, as well as the shortcomings of utilizing a multiphase oil-water salinity model for multiphase mixtures that are comprised of sand and water (and even including gas as well).

In some cases, the salinity manager 226 determines a volume fraction of the sand in the sand-water phase. For example, as discussed above, the multiphase oil-water salinity model and the multiphase sand-water salinity model are derived, generated, and/or based on Bruggeman's mixing formulas. In some embodiments, the salinity manager 226 may utilize the sand permittivity value (e.g., the sand permittivity offset) and/or the sand conductivity value (e.g., 0) in order to determine the volume fraction of the sand phase. For example, the salinity manager 226 may identify the conductivity values and/or permittivity values for the sand phase (e.g., sand point), water phase (e.g., water point), and for the mixture (e.g., active values) and may accordingly identify the fractional volume of the sand in the mixture and/or the factional volume of the water in the mixture.

As mentioned above, the multiphase flowrate system 220 may be implemented for determining one or more flowrates of a flow of a multiphase mixture, including individual flowrates of constituent phases of the multiphase mixture. In some case, the multiphase flowrate system 220 may determine flowrates based on a multiphase attenuation model. For example, the multiphase attenuation model may relate gamma ray measurements to a chemical makeup of various phases of a multiphase mixture. For instance, based on high- and low-energy gamma ray measurements (attenuations), the multiphase attenuation model may relate a specific chemical makeup of the multiphase mixture to gamma ray measurements for various single-phase points. In some cases, the multiphase attenuation model may be represented as an attenuation triangle (or other n-sided shape).

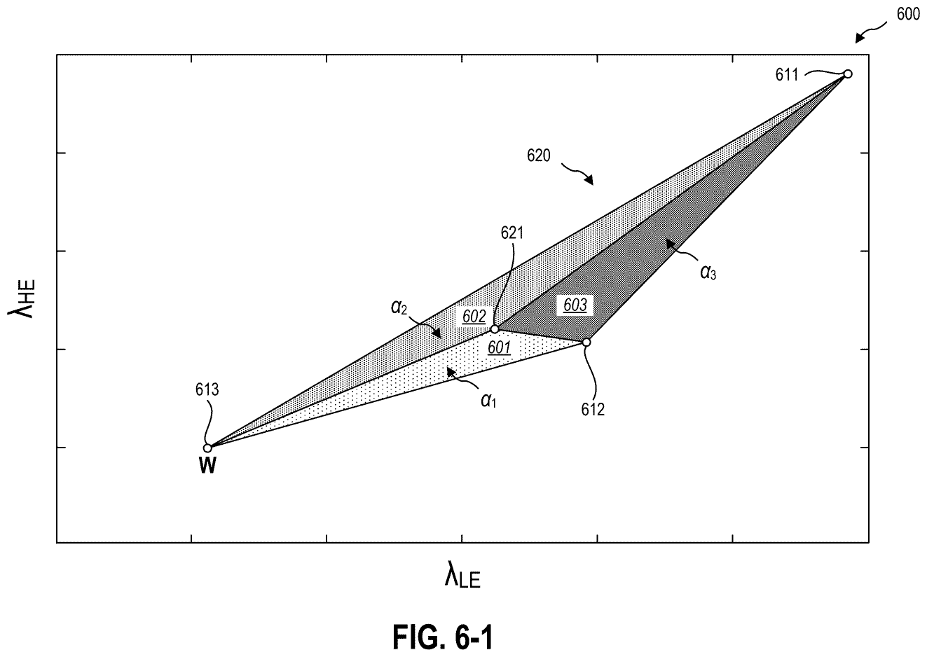

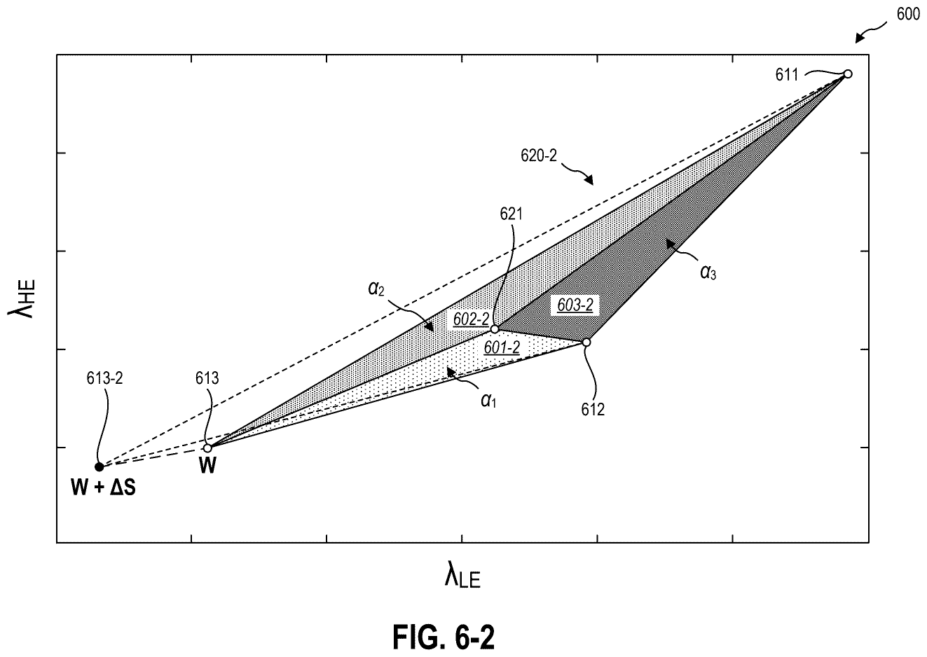

FIG. 6-1 illustrates an example multiphase attenuation model 600 for a multiphase mixture, according to at least one embodiment of the present disclosure. In some cases, the multiphase attenuation model 600 may be represented by an attenuation triangle. The multiphase attenuation model 600 may relate measured attenuation coefficients for high-energy gamma rays (λ_{HE}) and measured attenuation coefficients for low-energy gamma rays (λ_{LE}) of a multiphase mixture to the proportional makeup of the phases of the multiphase mixture in a (λ_{LE}, λ_{HE}) parameter space. For instance, λ_{LE} may correspond with a linear attenuation value or coefficient for low-energy gamma rays and λ_{HE} may correspond with a linear attenuation value or coefficient for high-energy gamma rays.

In some embodiments, the multiphase attenuation model 600 may be applicable to a 3-phase mixture. For example, the 3-phase mixture may include a first phase, a second phase, and a water phase. In some cases, a multiphase model in accordance with the techniques described herein may be associated with multiphase mixtures having any number of phases, such as 2-phase mixtures, 3-phase mixtures, 4-phase mixtures, or more. In some cases, the first phase may be a gas phase. In some examples, the second phase may be an oil phase or a sand phase. For instance, the multiphase mixture may be an oil-water-gas mixture, a sand-water-gas mixture, or mixture having other constituent phases.

The multiphase attenuation model 600 may include various single-phase points, representing the associated low- and high-energy gamma ray linear attenuation coefficients for mixtures of just that associated phase. For example, a first single-phase point 611 may correspond to the first phase and a second single-phase point 612 may correspond to the second phase. A water point 613 corresponds to the water phase. Defined between the various phase points may be a multiphase attenuation area 620. The multiphase attenuation area 620 may represent an area of potential gamma ray linear attenuation points (e.g., (λ_{LE}, λ_{HE}) ordered pair) which may be measured for the associated multiphase mixture.

The various single-phase points may be determined, measured, or established based on empirical data for the associated phase. For example, the λ_{LE} coefficient and the λ_{HE} coefficient may be determined or calculated for a single-phase point based on the density, ρ, of the phase. The linear coefficients, λ, may represent the fraction of gamma ray intensity that is absorbed or scattered by unit thickness of a phase. In some cases, the λ_{LE} linear attenuation coefficient and the λ_{HE} linear attenuation coefficient may be based on mass attenuation coefficients μ_{HE} and μ_{LE} for the phase. The mass attenuation coefficient, μ, may describe the attenuation per unit mass of the phase, for example, independent of density. For example, the linear attenuation coefficient, λ, may be determined for high- and low-energy based on the following formula:

$$λ = ρ \cdot μ$$

Accordingly, based on the multiphase area 620, and given a measured gamma ray attenuation point 621, the compositional makeup of the multiphase mixture may be determined. For example, from the measured gamma ray attenuation point 621, lines may be determined which extend to the first single-phase point 611, the second single-phase point 612, and the water point 613. Accordingly, the multiphase attenuation area 620 may be divided into a first phase attenuation area 601 corresponding with the first phase, a second phase attenuation area 602 corresponding with the second phase, and a water phase attenuation area 603 corresponding with the water phase. The ratio of the area of each phase attenuation area to the multiphase attenuation area 620 may represent the respective proportion of the associated phase in the multiphase mixture. Accordingly, based on taking high- and low-energy gamma ray measurements of the multiphase mixture, the fractional phase composition of the multiphase mixture may be determined with the multiphase attenuation model 600.

In some embodiments, the multiphase flowrate system 220 may determine a multiphase flowrate based on a multiphase attenuation model, such as the multiphase attenuation model 600. For example, the flowrate manager 230 may identify a volumetric flow rate of the multiphase mixture based on receiving a flowrate measurement from the flowrate sensor, such from a venturi sensor. Additionally, the flowrate manager 230 may receive high- and low-energy gamma ray attenuation measurements and may utilize the multiphase attenuation model to determine the proportional amounts of each phase in the multiphase mixture. Accordingly, the flowrate manager 230 may infer the respective flowrates of the phases based on the proportional makeup of the multiphase flow.

In some embodiments, the multiphase flowrate system 220 receives the multiphase attenuation model. For example, the multiphase attenuation model 600 may be established, known, and/or published for various different multiphase mixtures. The data manager 222 may accordingly access a library, system, or other resource for receiving a multiphase attenuation model corresponding to a multiphase mixture flowing in the wellbore.

As described herein, in some cases the salinity of the water phase may change during the flow of the multiphase mixture. For example, the fluid being circulated in the wellbore may interact and/or mix with a reservoir fluid, which may change the salinity of the resulting fluid that flows from the wellbore. In another example, fresh water may be introduced into the circulating fluid (e.g., either from injection into the wellbore or from interacting with an underground water source such as an aquifer), which may dilute and/or reduce the salinity. In another example, the circulating fluid may encounter a salt cap or salt formation, and additional salts may become dissolved in the fluid which may increase the salinity.

Accordingly, in these (and other) cases, the salinity of the multiphase mixture may change, which may affect the ability of the flowmeter to accurately determine the fractional composition of the mixture, and accordingly the phase flowrates. For instance, a change in salinity of the water phase may represent a change to the density of the water phase and/or a change to the effective mass attenuation coefficient of the water phase. Accordingly, the water point as represented by the multiphase attenuation model may be incorrect and/or may no longer accurately represent the actual (λ_{LE}, λ_{HE}) value of the water phase.

In some cases, the salinity calibration manager 228 may facilitate adjusting, updating, and/or generating a new multiphase attenuation model. For example, FIG. 6-2 illustrates an example of an updated multiphase attenuation model 600-2, according to at least one embodiment of the present disclosure.

The salinity calibration manager 228 may update the multiphase attenuation model based on adjusting the water point 613 and/or determining a new water point 613-2. For instance, the salinity calibration manager 228 may update the multiphase attenuation model based on identifying a change to the salinity of the multiphase mixture. In some cases, the salinity calibration manager 228 receives the updated salinity from the salinity manager 226. The salinity manager 226 may determine the updated salinity based on the techniques described above. For instance, the salinity manager 226 may utilize the multiphase sand-water salinity model to determine an updated salinity (e.g., for a sand-water mixture). In some cases, the salinity manager 226 utilizes the multiphase oil-water salinity model to determine updated salinity values (e.g., for oil-water mixtures).

Based on identifying a change to the salinity, the salinity calibration manager 228 may determine one or more of an updated density and/or updated mass attenuation coefficients for the water phase. For example, the salinity calibration manager may determine an updated density based on the properties of the salt in the water phase and its relative proportions. Similarly, the salinity calibration may determine the mass attenuation coefficient for the updated salinity of the water phase. For instance, various values may be known and/or established via empirical testing for the mass attenuation coefficients of water at a given salinity, and the salinity calibration manager may accordingly select, interpret, interpolate, and/or infer a mass attenuation coefficient for the identified salinity based on the empirical data.

Based on determining updated mass attenuation coefficients (e.g., for both high and low energy), the salinity calibration manager 228 may determine updated linear mass attenuation coefficients (λ_{LE}, λ_{HE}) to establish the updated water point 613-2. The updated water point 613-2 may corresponding with a shift and/or replotting of the water point from its original, initialized, and/or previous value (e.g., water point 613), to the updated position. Accordingly, based on the updated water point 613-2, the updated multiphase attenuation model 600-2 may be generated. For instance, based on shifting the water point as described, the updated multiphase attenuation model 600-2 may exhibit a corresponding updated attenuation area 620-2, along with corresponding updated phase areas 601-2, 602-2, and 603-2.

Accordingly, the flowrate manager 230 may accurately determine the multiphase flowrate of the multiphase mixture based on updates to the multiphase attenuation model accounting for changes in salinity of the water phase. For example, in some cases, the salinity of the water phase may change often and/or quite rapidly. Accordingly, the multiphase flowrate system 220 may identify changes in the salinity in real time, may update the multiphase attenuation model in real time, and may determine accurate, real-time flowrates of the various phases of the flow of the multiphase mixture.

In some cases, the mass attenuation coefficients and/or the density of the water phase may further be influenced by the salt species of the salts in the water phase. For example, in many cases, the salts in multiphase mixtures for wellbore applications may generally comprise (e.g., entirely or mostly) of sodium chloride (NaCl). Accordingly, in some cases, the detected changes in salinity and corresponding updates may be based on an assumption that the salinity of the water phase is entirely inclusive of NaCl. In some cases, such an assumption may be true (e.g., or at least result in substantially accurate multiphase flowrate determinations). For instance, in cases where a circulating fluid encounters a salt formation, which are typically formed of Halite (e.g., NaCl), the salinity of the water phase and/or changes thereto may typically be the result of NaCl in the water phase. Accordingly, in some cases the multiphase flowrate system 220 determines multiphase flowrates based on utilizing the chemical properties of Na and Cl ions dissolved in the water phase (e.g., for determining density and/or mass attenuation coefficients for the water phase).

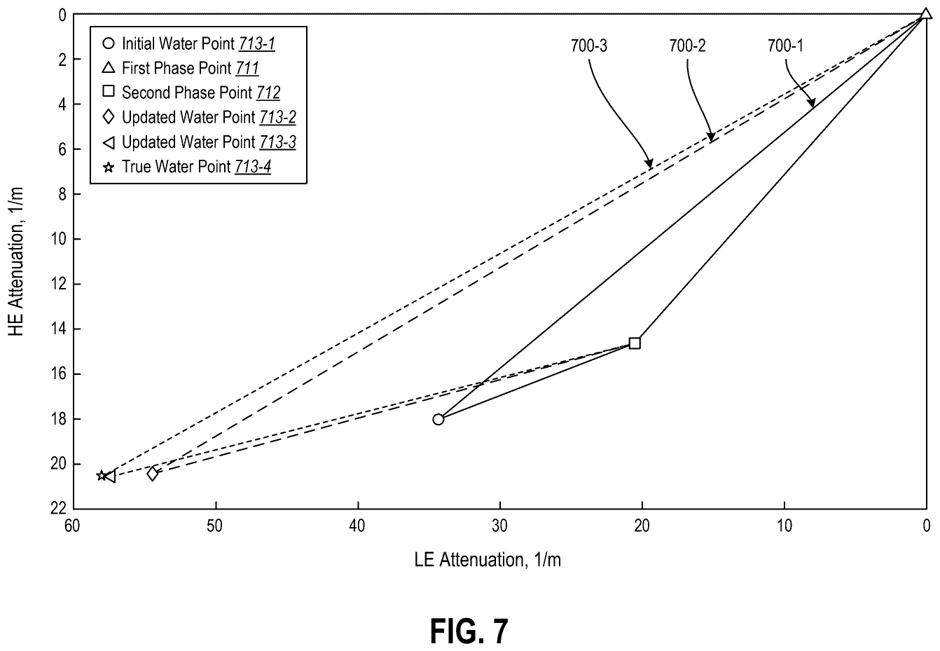

FIG. 7 illustrates an example of an updated multiphase attenuation model 700-3, according to at least one embodiment of the present disclosure. In FIG. 7, an original or initialized multiphase attenuation model 700-1 is represented, including a first phase point 711 corresponding to a first phase (e.g., gas) of a multiphase mixture, a second phase point 712 corresponding to a second phase (e.g., oil or sand) of a multiphase mixture, and an initial water point 713-1.

Based on identifying a change in salinity, the salinity calibration manager 228 may determine and updated water point, as described herein. Based on the assumption that the salt of the salinity is NaCl, a resulting updated water point 713-2 would be determined, and a corresponding updated multiphase attenuation model 700-2 generated. Accordingly, the updated multiphase attenuation model 700-2 may be associated with an NaCl salinity. As shown, the updated water point 713-2 is not entirely accurate to a true water point 713-4. For instance, the salinity in this case may be based on salt species other than NaCl and/or in addition to NaCl in the water phase. Accordingly, determining the updated water point 713-2 and generating the corresponding updated multiphase attenuation model 700-2 may result in inaccurate multiphase flowrates.

In some cases, the salinity calibration manager 228 may determine an updated water point 713-3 based on accounting for the salt species of the salts in the water phase. For example, as shown in FIG. 7, the updated water point 713-3 may be substantially accurate to the true water point 713-4. Accordingly, an updated multiphase attenuation model 700-3 may be generated which may more accurately reflect the attenuation properties of the multiphase mixture.

In some cases, the salinity calibration manager 228 may identify the salt species and their proportional makeup in the water phase based on an initial condition or state of the water phase. For example, during an initial period of the flow of the multiphase mixture, a sample of the multiphase fluid may be taken and analyzed to determine the chemical composition of the salts therein. This initial condition may be representative of a formation fluid. Accordingly, the salt species of the multiphase mixture may be determined, for example, for determining an initial water point.

Based on changes to the salinity, the salinity calibration manager 228 may determine or infer the salt species and their relative proportions which caused the change in the salinity. In some cases, the salinity calibration manager 228 does this based on identifying one or more operating conditions of the multiphase mixture circulating in the wellbore. For instance, the operating conditions may be based on user input identifying a specific scenario or use case in which the wellbore is being operated.

In some cases, an operating condition may correspond with the multiphase mixture encountering a salt formation or salt cap. For example, the salt cap may be primarily (or entirely) composed of an NaCl salt species. Accordingly, the salinity calibration manager 228 may infer that a determined change (e.g., increase) in salinity is resultant of an increase in the NaCl salt species in the multiphase mixture. Based on this assumption, and in connection with the knowledge of the initial salt species in the initial condition, the salinity calibration manager 228 may determine which salt species are present in the water phase and their respective proportions. Accordingly, the salinity calibration manager 228 may determine and updated density and/or mass attenuation coefficients for the water phase, and ultimately an updated multiphase attenuation model that is more accurate to the multiphase mixture.

In some cases, an operating condition may be associated with fresh water being introduced into the multiphase mixture. For example, fresh water may be injected into the wellbore, and/or the circulating fluid may encounter condensed water within the formation. Under such a condition, the salinity calibration manager 228 may infer that a change (e.g., decrease) in the salinity is due to the salts in the water phase being diluted, for example, in proportionate amounts. Accordingly, the salinity calibration manager 228 may assume that the relative proportions of the salt species from the initial condition remain substantially unchanged and may determine an updated water point.

In some cases, an operating condition may be associated with a brine being injected into the wellbore. For example, in some cases sea water, salt water from another source, or a created brine may be injected into the wellbore. Both the salinity and the salt species composition of the injected brine may be known and/or determined (e.g., measured). Based on identifying a change in the salinity of the multiphase mixture (e.g., flowing out of the wellbore), the salinity calibration manager 228 may determine the resulting salt species composition of the multiphase mixture. For example, based on the initial condition representing the salt species composition of the formation fluid, and based on the knowledge of the salt species composition of the injected brine (and associated salinity), the salinity calibration manager 228 may determine the resulting salt species composition of the multiphase mixture flowing from the wellbore based on a mixing of the formation fluid and the injected brine. The salinity calibration manager 228 may accordingly update the water point to reflect the determined salt species composition.

In this way, based on one or more operating conditions of the multiphase mixture, the salinity calibration manager 228 may identify a change in the salinity of the water phase, and may further identify the specific composition of the salt species in the water phase in order that an updated water point may be determined which more accurately reflects the multiphase mixture. For example, the updated water point may represent a more accurate approximation of the true water point than, for example, a water point as determined based on an assumption of only NaCl in the water phase.

In some cases, the salinity calibration manager 228 may determine one or more of mass attenuation coefficients, density, or an associated water point for the multiphase mixture based on assuming only NaCl in the water phase, for example, as an intermediate step. For example, in some cases, rather than directly updating the water point to the updated water point 713-3 from the initial water point 713-1, the salinity calibration manager 228 may instead determine the updated water point 713-2 and may apply a correction factor to the updated water point 713-2 to arrive at the updated water point 713-3. For instance, the salinity calibration manager 228 may determine the salt species composition of the multiphase mixture as described above, and may utilize the salt species composition to determine a correction factor which may be applied to the (λ_{LE}, λ_{HE}) values of the updated water point 713-2 (e.g., as determined based on assuming only NaCl) to determine the updated water point 713-3 (e.g., reflecting the actual salt species in the water phase). This intermediate step of implementing the NaCl assumption may be advantageous for determining a more accurate, updated water point 713-3 as described herein in connection with systems and/or flowmeters, for example, which may be calibrated and/or configured to take gamma ray measurements and determining multiphase flowrates based on the assumption that only NaCl is present in the water phase. For instance, some existing or conventional systems and/or flowmeters may operate in this way, and the multiphase flowrate system 220 may accordingly determine correction factors in this way in order to be implemented in connection with these existing systems.

In some cases, the correction factor may be based on a ratio of the (λ_{LE}, λ_{HE}) values based on the NaCl assumption to the (λ_{LE}, λ_{HE}) based on the salt species composition as determined above. Similarly, the correction factor may be based on a ration of (μ_{LE}, μ_{HE}) values as well. As an illustrative example, the salinity calibration manager 228 may determine the salt species composition of the water phase of a multiphase mixture flowing from the wellbore as shown in the tables below:

| | Cl | SO4 | Na | Ca | Mg | K | Sr |
|---|---|---|---|---|---|---|---|
| PPM | 82016 | 496 | 43168 | 6644 | 1025 | 830 | 475.1 |
| Weight % | 8.2016 | .0496 | 4.3168 | .6644 | .1025 | .083 | .04751 |

| | Weight Percent |
|---|---|
| NaCl | 10.97% |
| CaCl2 | 1.84% |
| MgCl2 | 0.40% |
| KCl | 0.23% |

Accordingly, based on established and/or measured empirical values for attenuation of the Na and Cl ions, the salinity calibration manager 228 may determine a λ_{LE} value of 54.4735 (1/m) and a λ_{HE} value of 20.2874 (1/m) for an assumption of only NaCl in the water phase. Further, based on established and/or measured empirical values for the attenuation of the various ions in the water phase and their relative proportions, the salinity calibration manager 228 may determine a λ_{LE} value of 57.65 (1/m) and a λ_{HE} value of 20.4646 (1/m) based on the specific salt species identified in the water phase. Accordingly, the salinity calibration manager 228 may determine a correction factor based on the ratio of the linear mass attenuation coefficients of the NaCl assumption to that of the identified salt species. In other words, the correction factor for low energy may be 54.4735/57.65=1.0602 and for high energy may be 20.2874/20.4646=1.0106.

In this way the correction factors may be applied to a (e.g., incorrect) water point as determined by an existing and/or conventional system or flowmeter and based on only NaCl accounting for salinity, and an accurate, updated water point may be determined in order that a multiphase attenuation model may be generated which more accurately reflects the attenuation properties of the multiphase mixture having a specific salt species composition therein. Accordingly, the multiphase flowrate system 220 may facilitate utilizing gamma ray measurements in order to accurately determine phase fraction flowrates of the various phases in a multiphase mixture while accounting for changes in salinity as well as the specific salt species present in the multiphase mixture.

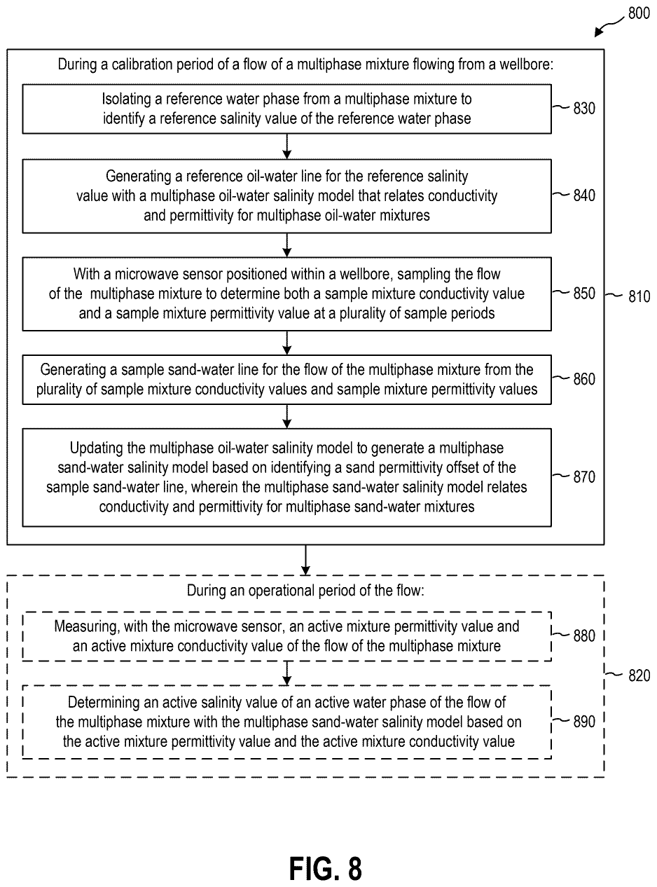

FIG. 8 illustrates a flow diagram for a method 800 or a series of acts for determining salinity of a flow of a multiphase mixture flowing from a wellbore as described herein, according to at least one embodiment of the present disclosure. While FIG. 8 illustrates acts according to one embodiment, alternative embodiments may add to, omit, reorder, or modify any of the acts of FIG. 8. In some cases, the method 800 is performed by a computer system. In some embodiments, the method 800 is performed as instructions stored on a computer-readable storage medium.

In some embodiments, the method 800 includes, at 810, performing various sub acts during a calibration period of the flow of a multiphase mixture.

In some embodiments, the method 800 includes an act 830 of isolating a reference water phase from a multiphase mixture to identify a reference salinity value of the reference water phase.

In some embodiments, the method 800 includes an act 840 of generating a reference oil-water line for the reference salinity value with a multiphase oil-water salinity model that relates conductivity and permittivity for multiphase oil-water mixtures.

In some embodiments, the method 800 includes an act 850 of, with a microwave sensor positioned within the wellbore, sampling the flow of the multiphase mixture to determine both a sample mixture conductivity value and a sample mixture permittivity value at a plurality of sample periods.

In some embodiments, the method 800 includes an act 860 of generating a sample sand-water line for the flow of the multiphase mixture from the plurality of sample mixture conductivity values and sample mixture permittivity values.

In some embodiments, the method 800 includes an act 870 of updating the multiphase oil-water salinity model to generate a multiphase sand-water salinity model based on identifying a sand permittivity offset of the sample sand-water line, wherein the multiphase sand-water salinity model relates conductivity and permittivity for multiphase sand-water mixtures.

In some embodiments, the method 800 includes, at 820, performing various sub acts during an operational period of the flow of the multiphase mixture. In some cases, the act 820 may be an optional act of the method 800.

In some embodiments, the method 800 includes an act 880 of measuring, with the microwave sensor, an active mixture permittivity value and an active mixture conductivity value of the flow of the multiphase mixture.

In some embodiments, the method 800 includes an act 890 of determining an active salinity value of an active water phase of the flow of the multiphase mixture with the multiphase sand-water salinity model based on the active mixture permittivity value and the active mixture conductivity value.

In some embodiments, the method 800 further includes, during the calibration period, determining a reference slope of the reference oil-water line, and generating the sample sand-water line is further based on applying the reference slope to the plurality of sample mixture conductivity values and sample mixture permittivity values.

In some embodiments, the multiphase oil-water salinity model relates to multiphase oil-water mixtures that are substantially water continuous, and the ratio of the oil-water mixture conductivity to the oil-water mixture permittivity is assumed to be approximately equal to the ratio of the water conductivity to the water permittivity.

In some embodiments, the multiphase sand-water salinity model relates to multiphase sand-water mixtures that are substantially water continuous, and the ratio of the sand-water mixture conductivity to the sand-water mixture permittivity is assumed to be offset from the ratio of the water conductivity of the water permittivity by the sand permittivity offset.

In some embodiments, the sand permittivity offset is assumed to be constant for sand-water mixtures of varying salinity.

In some embodiments, the multiphase oil-water salinity model defines theoretical water conductivities for input mixture permittivities and input mixture conductivities.

In some embodiments, the multiphase oil-water salinity model is further based on a pressure, a temperature, and a microwave frequency for the input mixture permittivity values and the input mixture conductivity values.

In some embodiments, the microwave sensor takes permittivity and conductivity measurements at a frequency between 1 and 3 GHz, optionally between 1.9 and 2.1 GHz, for instance approximately 2 GHz.

In some embodiments, the slope of the reference oil-water line and the sample sand-water line is approximately equal.

In some embodiments, identifying the reference salinity value includes measuring the density of the reference water phase.

In some embodiments, identifying the reference salinity value includes measuring a reference conductivity with the microwave sensor based on flowing the isolated reference water phase to the microwave sensor.

In some embodiments, determining the active salinity includes estimating an active water conductivity of the active water phase with the multiphase sand-water salinity model based on the active mixture permittivity and the active mixture conductivity.

In some embodiments, the method 800 further includes determining a mass attenuation coefficient for the active water phase based on the active salinity and determining a flowrate of the active water phase based on the mass attenuation coefficient.

In some embodiments, the method 800 further includes determining, based on the sample sand-water line, a sand permittivity value of a sand point of the multiphase mixture, determining a water permittivity value with the multiphase sand-water salinity model based on the active salinity value, and determining an active sand fraction and an active water fraction of the multiphase mixture based on the sand permittivity value, the water permittivity value, and the active mixture permittivity.

In some embodiments, the method 800 further includes determining the active sand fraction and the active water fraction of the multiphase mixture based on Bruggeman's mixing formula.

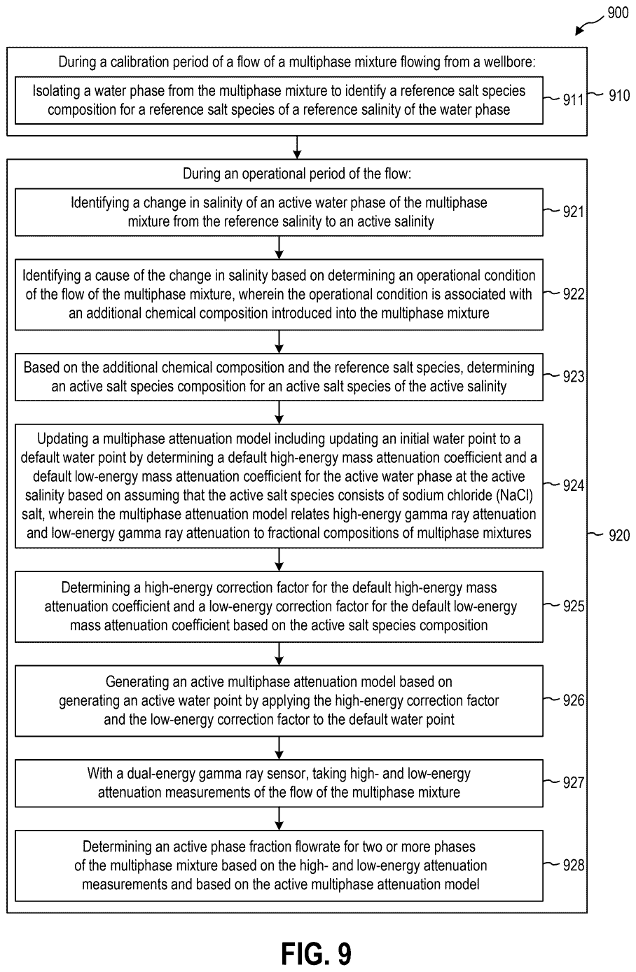

FIG. 9 illustrates a flow diagram for a method 900 or a series of acts for determining salinity of a flow of a multiphase mixture flowing from a wellbore as described herein, according to at least on embodiment of the present disclosure. While FIG. 9 illustrates acts according to one embodiment, alternative embodiments may add to, omit, reorder, or modify any of the acts of FIG. 9. In some cases, the method 900 is performed by a computer system. In some embodiments, the method 900 is performed as instructions stored on a computer-readable storage medium.

In some embodiments, the method 900 includes, at 910, performing one or more acts during a calibration period of a flow of a multiphase mixture flowing from a wellbore.

In some embodiments, the method 900 includes an act 911 of isolating a water phase from the multiphase mixture to identify a reference salt species composition for a reference salt species of a reference salinity of the water phase.

In some embodiments, the method 900 includes, at 920, performing one or more acts during an operational period of the flow of the multiphase mixture flowing from the wellbore.

In some embodiments, the method 900 includes an act 921 of identifying a change in salinity of an active water phase of the multiphase mixture from the reference salinity to an active salinity.

In some embodiments, the method 900 includes an act 922 of identifying a cause of the change in salinity based on determining an operational condition of the flow of the multiphase mixture, wherein the operational condition is associated with an additional chemical composition introduced into the multiphase mixture.

In some embodiments, the method 900 includes an act 923 of based on the additional chemical composition and the reference salt species, determining an active salt species composition for an active salt species of the active salinity.

In some embodiments, the method 900 includes an act 924 of updating a multiphase attenuation model including updating an initial water point to a default water point by determining a default high-energy mass attenuation coefficient and a default low-energy mass attenuation coefficient for the active water phase at the active salinity based on assuming that the active salt species consists of sodium chloride (NaCl) salt, wherein the multiphase attenuation model relates high-energy gamma ray attenuation and low-energy gamma ray attenuation to fractional compositions of multiphase mixtures.

In some embodiments, the method 900 includes an act 925 of determining a high-energy correction factor for the default high-energy mass attenuation coefficient and a low-energy correction factor for the default low-energy mass attenuation coefficient based on the active salt species composition.

In some embodiments, the method 900 includes an act 926 of generating an active multiphase attenuation model based on generating an active water point by applying the high-energy correction factor and the low-energy correction factor to the default water point.

In some embodiments, the method 900 includes an act 927 of with a dual-energy gamma ray sensor, taking high- and low-energy attenuation measurements of the flow of the multiphase mixture.

In some embodiments, the method 900 includes an act 928 of determining an active phase fraction flowrate for two or more phases of the multiphase mixture based on the high- and low-energy attenuation measurements and based on the active multiphase attenuation model.

In some embodiments, the additional chemical composition is one or more of fresh water injected into the wellbore, fresh water introduced into the multiphase mixture from a formation, a saltwater brine injected into the wellbore, or NaCl salt introduced into the multiphase mixture from a salt formation.

In some embodiments, the active salt species includes one or more salts in addition to NaCl salt.

In some embodiments, identifying the change in salinity and determining the active phase fraction flowrate is performed in real time.

In some embodiments, the methods 800 and 900 may be combined, for example, to both determine salinity in accordance with the method 800 and to determine multiphase flowrates based on the determined salinity in accordance with the method 900.

For instance, in some cases a method is described herein for determining multiphase flowrates of a flow of a multiphase mixture flowing from a wellbore.

In some embodiments, various acts are performed during a calibration period of the flow of the multiphase mixture. In some embodiments, the method includes isolating a reference water phase from the multiphase mixture to identify a reference salinity of the reference water phase including identifying a reference salt species composition for a reference salt species of the reference salinity. The method may include generating a reference oil-water line for the reference salinity with a multiphase oil-water salinity model that relates conductivity and permittivity for multiphase oil-water mixtures. In some cases, the method includes with a microwave sensor positioned within the wellbore, sampling the flow of the multiphase mixture to determine both a sample mixture conductivity value and a sample mixture permittivity value at a plurality of sample periods. In some cases, the method includes generating a sample sand-water line for the flow of the multiphase mixture from the plurality of sample mixture conductivity values and sample mixture permittivity values. In some examples, the method includes updating the multiphase oil-water salinity model to generate a multiphase sand-water salinity model based on identifying a sand permittivity offset of the sample sand-water line, wherein the multiphase sand-water salinity model relates conductivity and permittivity for multiphase sand-water mixtures.

In some embodiments, various acts are performed during an operational period of the flow of the multiphase mixture. For example, in some cases the method includes measuring, with the microwave sensor, an active mixture permittivity value and an active mixture conductivity value of the flow of the multiphase mixture. In some instances, the method includes determining an active salinity of an active water phase of the flow of the multiphase mixture with the multiphase sand-water salinity model based on the active mixture permittivity value and the active mixture conductivity value. The method can include identifying an additional chemical composition introduced into the multiphase mixture resulting in the active salinity. The method may include, based on the additional chemical composition and the reference salt species, determining an active salt species composition for an active salt species of the active salinity. In some cases, the method includes updating a multiphase attenuation model including updating an initial water point to a default water point by determining a default high-energy mass attenuation coefficient and a default low-energy mass attenuation coefficient for the active water phase at the active salinity based on assuming that the active salt species consists of sodium chloride (NaCl) salt, wherein the multiphase attenuation model relates high-energy gamma ray attenuation and low-energy gamma ray attenuation to fractional compositions of multiphase mixtures. The method may include determining a high-energy correction factor for the default high-energy mass attenuation coefficient and a low-energy correction factor for the default low-energy mass attenuation coefficient based on the active salt species composition. In various instance the method includes generating an active multiphase attenuation model based on generating an active water point by applying the high-energy correction factor and the low-energy correction factor to the default water point. The method may include, with a dual-energy gamma ray sensor, taking high- and low-energy attenuation measurements of the flow of the multiphase mixture. The method may also include determining an active phase fraction flowrate for two or more phases of the multiphase mixture based on the high- and low-energy attenuation measurements and based on the active multiphase attenuation model.

Turning now to FIG. 10, this figure illustrates certain components that may be included within a computer system 1000. One or more computer systems 1000 may be used to implement the various devices, components, and systems described herein.

The computer system 1000 includes a processor 1001. The processor 1001 may be a general-purpose single- or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 1001 may be referred to as a central processing unit (CPU). Although just a single processor 1001 is shown in the computer system 1000 of FIG. 10, in an alternative configuration, a combination of processors (e.g., an ARM and DSP) could be used.

The computer system 1000 also includes memory 1003 in electronic communication with the processor 1001. The memory 1003 may include computer-readable storage media and can be any available media that can be accessed by a general purpose or special purpose computer system. Computer-readable media that store computer-executable instructions are non-transitory computer-readable media (device). Computer-readable media that carry computer-executable instructions are transmission media. Thus, by way of example and not limitations, embodiment of the present disclosure can comprise at least two distinctly different kinds of computer-readable media: non-transitory computer-readable media (devices) and transmission media.

Both non-transitory computer-readable media (devices) and transmission media may be used temporarily to store or carry software instructions in the form of computer readable program code that allows performance of embodiments of the present disclosure. Non-transitory computer-readable media may further be used to persistently or permanently store such software instructions. Examples of non-transitory computer-readable storage media include physical memory (e.g., RAM, ROM, EPROM, EEPROM, etc.), optical disk storage (e.g., CD, DVD, HDDVD, Blu-ray, etc.), storage devices (e.g., magnetic disk storage, tape storage, diskette, etc.), flash or other solid-state storage or memory, or any other non-transmission medium which can be used to store program code in the form of computer-executable instructions or data structures and which can be accessed by a general purpose or special purpose computer, whether such program code is stored or in software, hardware, firmware, or combinations thereof.

Instructions 1005 and data 1007 may be stored in the memory 1003. The instructions 1005 may be executable by the processor 1001 to implement some or all of the functionality disclosed herein. Executing the instructions 1005 may involve the use of the data 1007 that is stored in the memory 1003. Any of the various examples of modules and components described herein may be implemented, partially or wholly, as instructions 1005 stored in memory 1003 and executed by the processor 1001. Any of the various examples of data described herein may be among the data 1007 that is stored in memory 1003 and used during execution of the instructions 1005 by the processor 1001.

A computer system 1000 may also include one or more communication interfaces 1009 for communicating with other electronic devices. The communication interface(s) 1009 may be based on wired communication technology, wireless communication technology, or both. Some examples of communication interfaces 1009 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

The communication interfaces 1009 may connect the computer system 1000 to a network. A “network” or “communications network” may generally be defined as one or more data links that enable the transport of electronic data between computer systems and/or modules, engines, or other electronic devices, or combinations thereof. When information is transferred or provided over a communication network or another communications connection (either hardwired, wireless, or a combination of hardwired or wireless) to a computing device, the computing device properly views the connection as a transmission medium. Transmission media can include a communication network and/or data links, carrier waves, wireless signals, and the like, which can be used to carry desired program or template code means or instructions in the form of computer-executable instruction or data structures and which can be accessed by a general purpose or special purpose computer.

A computer system 1000 may also include one or more input devices 1011 and one or more output devices 1013. Some examples of input devices 1011 include a keyboard, mouse, microphone, remote control device, button, joystick, trackball, touchpad, and lightpen. Some examples of output devices 1013 include a speaker and a printer. One specific type of output device that is typically included in a computer system 1000 is a display device 1015. Display devices 1015 used with embodiments disclosed herein may utilize any suitable image projection technology, such as liquid crystal display (LCD), light-emitting diode (LED), gas plasma, electroluminescence, or the like. A display controller 1017 may also be provided, for converting data 1007 stored in the memory 1003 into one or more of text, graphics, or moving images (as appropriate) shown on the display device 1015.

The various components of the computer system 1000 may be coupled together by one or more buses, which may include one or more of a power bus, a control signal bus, a status signal bus, a data bus, other similar components, or combinations thereof. For the sake of clarity, the various buses are illustrated in FIG. 10 as a bus system 1019.

The techniques described herein may be implemented in hardware, software, firmware, or any combination thereof, unless specifically described as being implemented in a specific manner. Any features described as modules, components, or the like may also be implemented together in an integrated logic device or separately as discrete but interoperable logic devices. If implemented in software, the techniques may be realized at least in part by a non-transitory processor-readable storage medium comprising instructions that, when executed by at least one processor, perform one or more of the methods described herein. The instructions may be organized into routines, programs, objects, components, data structures, etc., which may perform particular tasks and/or implement particular data types, and which may be combined or distributed as desired in various embodiments.

Further, upon reaching various computer system components, program code in the form of computer-executable instructions or data structures can be transferred automatically or manually from transmission media to non-transitory computer-readable storage media (or vice versa). For example, computer executable instructions or data structures received over a network or data link can be buffered in memory (e.g., RAM) within a network interface module (NIC), and then eventually transferred to computer system RAM and/or to less volatile non-transitory computer-readable storage media at a computer system. Thus, it should be understood that non-transitory computer-readable storage media can be included in computer system components that also (or even primarily) utilize transmission media.

### INDUSTRIAL APPLICABILITY

The following description from section A1 to section C2 includes various embodiments that, where feasible, may be combined in any permutation. For example, the embodiment of section A1 may be combined with any or all embodiments of the following paragraphs. Embodiments that describe acts of a method may be combined with embodiments that describe, for example, systems and/or devices. Any permutation of the following paragraphs is considered to be hereby disclosed for the purposes of providing “unambiguously derivable support” for any claim amendment based on the following paragraphs. Furthermore, the following paragraphs provide support such that any combination of the following paragraphs would not create an “intermediate generalization.”
A1. A method of calibrating a multiphase flowmeter for determining salinity of a flow of a multiphase mixture flowing from a wellbore, comprising, during a calibration period of the flow:
   isolating a reference water phase from the multiphase mixture to identify a reference salinity value of the reference water phase;
   generating a reference oil-water line for the reference salinity value with a multiphase oil-water salinity model that relates conductivity and permittivity for multiphase oil-water mixtures;
   with a microwave sensor positioned within the wellbore, sampling the flow of the multiphase mixture to determine both a sample mixture conductivity value and a sample mixture permittivity value at a plurality of sample periods;
   generating a sample sand-water line for the flow of the multiphase mixture from the plurality of sample mixture conductivity values and sample mixture permittivity values; and
   updating the multiphase oil-water salinity model to generate a multiphase sand-water salinity model based on identifying a sand permittivity offset of the sample sand-water line, wherein the multiphase sand-water salinity model relates conductivity and permittivity for multiphase sand-water mixtures.
A2. The method of A1, further comprising, determining a reference slope of the reference oil-water line, and generating the sample sand-water line is further based on applying the reference slope to the plurality of sample mixture conductivity values and sample mixture permittivity values.
A3. The method of A1 or A2, wherein the multiphase oil-water salinity model relates to multiphase oil-water mixtures that are substantially water continuous, and a ratio of the oil-water mixture conductivity to the oil-water mixture permittivity is assumed to be approximately equal to a ratio of the water conductivity to the water permittivity.
A4. The method of A3, wherein the multiphase sand-water salinity model relates to multiphase sand-water mixtures that are substantially water continuous, and a ratio of the sand-water mixture conductivity to the sand-water mixture permittivity is assumed to be offset from a ratio of the water conductivity of the water permittivity by the sand permittivity offset, and the sand permittivity offset is assumed to be constant for sand-water mixtures of varying salinity.
A5. The method of any of A1-A4, wherein the multiphase oil-water salinity model defines theoretical water conductivities for input mixture permittivity values and input mixture conductivity values.
A6. The method of A5, wherein the multiphase oil-water salinity model is further based on a pressure, a temperature, and a microwave frequency for the input mixture permittivity values and the input mixture conductivity values.
A7. The method of A5 or A6, wherein the microwave sensor takes permittivity and conductivity measurements at a frequency between 1 and 3 GHz.
A8. The method of any of A1-A6, wherein a slope of the reference oil-water line and the sample sand-water line is approximately equal.
A9. The method of any of A1-A8, wherein identifying the reference salinity value includes measuring density of the reference water phase.
A10. The method of any of A1-A9, wherein identifying the reference salinity value includes measuring a reference conductivity with the microwave sensor based on flowing the isolated reference water phase to the microwave sensor.
B1. A method of determining salinity of a flow of a multiphase mixture flowing from a wellbore, comprising:
   during a calibration period of the flow:
      isolating a reference water phase from the multiphase mixture to identify a reference salinity value of the reference water phase;
      generating a reference oil-water line for the reference salinity value with a multiphase oil-water salinity model that relates conductivity and permittivity for multiphase oil-water mixtures;
      with a microwave sensor positioned within the wellbore, sampling the flow of the multiphase mixture to determine both a sample mixture conductivity value and a sample mixture permittivity value at a plurality of sample periods;
      generating a sample sand-water line for the flow of the multiphase mixture from the plurality of sample mixture conductivity values and sample mixture permittivity values; and
      updating the multiphase oil-water salinity model to generate a multiphase sand-water salinity model based on identifying a sand permittivity offset of the sample sand-water line, wherein the multiphase sand-water salinity model relates conductivity and permittivity for multiphase sand-water mixtures; and
   during an operational period of the flow:
      measuring, with the microwave sensor, an active mixture permittivity value and an active mixture conductivity value of the flow of the multiphase mixture; and
      determining an active salinity value of an active water phase of the flow of the multiphase mixture with the multiphase sand-water salinity model based on the active mixture permittivity value and the active mixture conductivity value.
B2. The method of B1, wherein determining the active salinity value includes estimating an active water conductivity of the active water phase with the multiphase sand-water salinity model based on the active mixture permittivity value and the active mixture conductivity value.
B3. The method of B1 or B2, further comprising:
   determining a mass attenuation coefficient for the active water phase based on the active salinity value; and
   determining a flowrate of the active water phase based on the mass attenuation coefficient.
B4. The method of any of B1-B3, further comprising:
   determining, based on the sample sand-water line, a sand permittivity value of a sand point of the multiphase mixture;
   determining a water permittivity value with the multiphase sand-water salinity model based on the active salinity value; and
   determining an active sand fraction and an active water fraction of the multiphase mixture based on the sand permittivity value, the water permittivity value, and the active mixture permittivity value.
B15. The method of claim B4, further comprising determining the active sand fraction and the active water fraction of the multiphase mixture based on Bruggeman's mixing formula.
C1. A method of determining multiphase flowrates of a flow of a multiphase mixture flowing from a wellbore, comprising:
   during a calibration period of the flow:
      isolating a water phase from the multiphase mixture to identify a reference salt species composition for a reference salt species of a reference salinity of the water phase; and
   during an operational period of the flow:
      identifying a change in salinity of an active water phase of the multiphase mixture from the reference salinity to an active salinity;
      identifying a cause of the change in salinity based on determining an operational condition of the flow of the multiphase mixture, wherein the operational condition is associated with an additional chemical composition introduced into the multiphase mixture;
      based on the additional chemical composition and the reference salt species, determining an active salt species composition for an active salt species of the active salinity;
      updating a multiphase attenuation model including updating an initial water point to a default water point by determining a default high-energy mass attenuation coefficient and a default low-energy mass attenuation coefficient for the active water phase at the active salinity based on assuming that the active salt species consists of sodium chloride (NaCl) salt, wherein the multiphase attenuation model relates high-energy gamma ray attenuation and low-energy gamma ray attenuation to fractional compositions of multiphase mixtures;
      determining a high-energy correction factor for the default high-energy mass attenuation coefficient and a low-energy correction factor for the default low-energy mass attenuation coefficient based on the active salt species composition;
      generating an active multiphase attenuation model based on generating an active water point by applying the high-energy correction factor and the low-energy correction factor to the default water point;
      with a dual-energy gamma ray sensor, taking high- and low-energy attenuation measurements of the flow of the multiphase mixture; and
      determining an active phase fraction flowrate for two or more phases of the multiphase mixture based on the high- and low-energy attenuation measurements and based on the active multiphase attenuation model.
C2 The method of C1, wherein the additional chemical composition is one or more of:
   fresh water injected into the wellbore;
   fresh water introduced into the multiphase mixture from a formation;
   a saltwater brine injected into the wellbore; or
   NaCl salt introduced into the multiphase mixture from a salt formation.
C3. The method of C1 or C2, wherein the active salt species includes one or more salts in addition to NaCl salt.
C4. The method of any of C1-C3, wherein identifying the change in salinity and determining the active phase fraction flowrate is performed in real time.
D1. A method of determining multiphase flowrates of a flow of a multiphase mixture flowing from a wellbore, comprising:
   during a calibration period of the flow:
      isolating a reference water phase from the multiphase mixture to identify a reference salinity of the reference water phase including identifying a reference salt species composition for a reference salt species of the reference salinity;
      generating a reference oil-water line for the reference salinity with a multiphase oil-water salinity model that relates conductivity and permittivity for multiphase oil-water mixtures;
      with a microwave sensor positioned within the wellbore, sampling the flow of the multiphase mixture to determine both a sample mixture conductivity value and a sample mixture permittivity value at a plurality of sample periods;
      generating a sample sand-water line for the flow of the multiphase mixture from the plurality of sample mixture conductivity values and sample mixture permittivity values; and
      updating the multiphase oil-water salinity model to generate a multiphase sand-water salinity model based on identifying a sand permittivity offset of the sample sand-water line, wherein the multiphase sand-water salinity model relates conductivity and permittivity for multiphase sand-water mixtures; and
   during an operational period of the flow:
      measuring, with the microwave sensor, an active mixture permittivity value and an active mixture conductivity value of the flow of the multiphase mixture;
      determining an active salinity of an active water phase of the flow of the multiphase mixture with the multiphase sand-water salinity model based on the active mixture permittivity value and the active mixture conductivity value;
      identifying an additional chemical composition introduced into the multiphase mixture resulting in the active salinity;
      based on the additional chemical composition and the reference salt species, determining an active salt species composition for an active salt species of the active salinity;
      updating a multiphase attenuation model including updating an initial water point to a default water point by determining a default high-energy mass attenuation coefficient and a default low-energy mass attenuation coefficient for the active water phase at the active salinity based on assuming that the active salt species consists of sodium chloride (NaCl) salt, wherein the multiphase attenuation model relates high-energy gamma ray attenuation and low-energy gamma ray attenuation to fractional compositions of multiphase mixtures;
      determining a high-energy correction factor for the default high-energy mass attenuation coefficient and a low-energy correction factor for the default low-energy mass attenuation coefficient based on the active salt species composition;
      generating an active multiphase attenuation model based on generating an active water point by applying the high-energy correction factor and the low-energy correction factor to the default water point;
      with a dual-energy gamma ray sensor, taking high- and low-energy attenuation measurements of the flow of the multiphase mixture; and
      determining an active phase fraction flowrate for two or more phases of the multiphase mixture based on the high- and low-energy attenuation measurements and based on the active multiphase attenuation model.
D2. A system, comprising:
   a processor;
   memory in electronic communication with the processor; and
   instructions stored in the memory, the instructions being executable by the processor to perform the method of D1.
D3. A computer-readable storage medium including instructions which, when executed by a processor, cause the processor to perform the method of D1.

The embodiments of the multiphase flowrate have been primarily described with reference to wellbore drilling operations; the multiphase flowrate described herein may be used in applications other than the drilling of a wellbore. In other embodiments, the multiphase flowrate according to the present disclosure may be used outside a wellbore or other downhole environment used for the exploration or production of natural resources. For instance, the multiphase flowrate of the present disclosure may be used in a borehole used for placement of utility lines. Accordingly, the terms “wellbore,” “borehole” and the like should not be interpreted to limit tools, systems, assemblies, or methods of the present disclosure to any particular industry, field, or environment.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that is within standard manufacturing or process tolerances, or which still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements. Additionally, as used herein, the term “and/or” includes any and all combinations of one or more of the associated listed items.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A method of calibrating a multiphase flowmeter for determining salinity of a flow of a multiphase mixture flowing from a wellbore, comprising, during a calibration period of the flow:
isolating a reference water phase from the multiphase mixture to identify a reference salinity value of the reference water phase;
generating a reference oil-water line for the reference salinity value with a multiphase oil-water salinity model that relates conductivity and permittivity for multiphase oil-water mixtures;
with a microwave sensor positioned within the wellbore, sampling the flow of the multiphase mixture to determine both a sample mixture conductivity value and a sample mixture permittivity value at a plurality of sample periods;
generating a sample sand-water line for the flow of the multiphase mixture from the plurality of sample mixture conductivity values and sample mixture permittivity values; and
updating the multiphase oil-water salinity model to generate a multiphase sand-water salinity model based on identifying a sand permittivity offset of the sample sand-water line, wherein the multiphase sand-water salinity model relates conductivity and permittivity for multiphase sand-water mixtures.

2. The method of claim 1, further comprising, determining a reference slope of the reference oil-water line, and generating the sample sand-water line is further based on applying the reference slope to the plurality of sample mixture conductivity values and sample mixture permittivity values.

3. The method of claim 1, wherein the multiphase oil-water salinity model relates to multiphase oil-water mixtures that are substantially water continuous, and a ratio of the oil-water mixture conductivity to the oil-water mixture permittivity is assumed to be approximately equal to a ratio of the water conductivity to the water permittivity.

4. The method of claim 3, wherein the multiphase sand-water salinity model relates to multiphase sand-water mixtures that are substantially water continuous, and a ratio of the sand-water mixture conductivity to the sand-water mixture permittivity is assumed to be offset from a ratio of the water conductivity of the water permittivity by the sand permittivity offset, and the sand permittivity offset is assumed to be constant for sand-water mixtures of varying salinity.

5. The method of claim 1, wherein the multiphase oil-water salinity model defines theoretical water conductivities for input mixture permittivity values and input mixture conductivity values.

6. The method of claim 5, wherein the multiphase oil-water salinity model is further based on a pressure, a temperature, and a microwave frequency for the input mixture permittivity values and the input mixture conductivity values.

7. The method of claim 5, wherein the microwave sensor takes permittivity and conductivity measurements at a frequency between 1 and 3 GHz.

8. The method of claim 1, wherein a slope of the reference oil-water line and the sample sand-water line is approximately equal.

9. The method of claim 1, wherein identifying the reference salinity value includes measuring density of the reference water phase.

10. The method of claim 1, wherein identifying the reference salinity value includes measuring a reference conductivity with the microwave sensor based on flowing the isolated reference water phase to the microwave sensor.

11. A method of determining salinity of a flow of a multiphase mixture flowing from a wellbore, comprising:
during a calibration period of the flow:
isolating a reference water phase from the multiphase mixture to identify a reference salinity value of the reference water phase;
generating a reference oil-water line for the reference salinity value with a multiphase oil-water salinity model that relates conductivity and permittivity for multiphase oil-water mixtures;
with a microwave sensor positioned within the wellbore, sampling the flow of the multiphase mixture to determine both a sample mixture conductivity value and a sample mixture permittivity value at a plurality of sample periods;
generating a sample sand-water line for the flow of the multiphase mixture from the plurality of sample mixture conductivity values and sample mixture permittivity values; and
updating the multiphase oil-water salinity model to generate a multiphase sand-water salinity model based on identifying a sand permittivity offset of the sample sand-water line, wherein the multiphase sand-water salinity model relates conductivity and permittivity for multiphase sand-water mixtures; and
during an operational period of the flow:
measuring, with the microwave sensor, an active mixture permittivity value and an active mixture conductivity value of the flow of the multiphase mixture; and
determining an active salinity value of an active water phase of the flow of the multiphase mixture with the multiphase sand-water salinity model based on the active mixture permittivity value and the active mixture conductivity value.

12. The method of claim 11, wherein determining the active salinity value includes estimating an active water conductivity of the active water phase with the multiphase sand-water salinity model based on the active mixture permittivity value and the active mixture conductivity value.

13. The method of claim 11, further comprising:
determining a mass attenuation coefficient for the active water phase based on the active salinity value; and
determining a flowrate of the active water phase based on the mass attenuation coefficient.

14. The method of claim 11, further comprising:
determining, based on the sample sand-water line, a sand permittivity value of a sand point of the multiphase mixture;
determining a water permittivity value with the multiphase sand-water salinity model based on the active salinity value; and
determining an active sand fraction and an active water fraction of the multiphase mixture based on the sand permittivity value, the water permittivity value, and the active mixture permittivity value.

15. The method of claim 14, further comprising determining the active sand fraction and the active water fraction of the multiphase mixture based on Bruggeman's mixing formula.

16. A method of determining multiphase flowrates of a flow of a multiphase mixture flowing from a wellbore, comprising:
during a calibration period of the flow:
isolating a water phase from the multiphase mixture to identify a reference salt species composition for a reference salt species of a reference salinity of the water phase; and
during an operational period of the flow:
identifying a change in salinity of an active water phase of the multiphase mixture from the reference salinity to an active salinity;
identifying a cause of the change in salinity based on determining an operational condition of the flow of the multiphase mixture, wherein the operational condition is associated with an additional chemical composition introduced into the multiphase mixture;
based on the additional chemical composition and the reference salt species, determining an active salt species composition for an active salt species of the active salinity;
updating a multiphase attenuation model including updating an initial water point to a default water point by determining a default high-energy mass attenuation coefficient and a default low-energy mass attenuation coefficient for the active water phase at the active salinity based on assuming that the active salt species consists of sodium chloride (NaCl) salt, wherein the multiphase attenuation model relates high-energy gamma ray attenuation and low-energy gamma ray attenuation to fractional compositions of multiphase mixtures;
determining a high-energy correction factor for the default high-energy mass attenuation coefficient and a low-energy correction factor for the default low-energy mass attenuation coefficient based on the active salt species composition;
generating an active multiphase attenuation model based on generating an active water point by applying the high-energy correction factor and the low-energy correction factor to the default water point;
with a dual-energy gamma ray sensor, taking high- and low-energy attenuation measurements of the flow of the multiphase mixture; and
determining an active phase fraction flowrate for two or more phases of the multiphase mixture based on the high- and low-energy attenuation measurements and based on the active multiphase attenuation model.

17. The method of claim 16, wherein the additional chemical composition is one or more of:
fresh water injected into the wellbore;
fresh water introduced into the multiphase mixture from a formation;
a saltwater brine injected into the wellbore; or
NaCl salt introduced into the multiphase mixture from a salt formation.

18. The method of claim 16, wherein the active salt species includes one or more salts in addition to NaCl salt.

19. The method of claim 16, wherein identifying the change in salinity and determining the active phase fraction flowrate is performed in real time.
